(19) United States
(12) Patent Application Publication
(10) Pub. No.: US 2005/0182005 A1
(43) Pub. Date: Aug. 18, 2005

(54) Anti-microRNA oligonucleotide molecules

(76) Inventors: Thomas H. Tuschl, New York, NY (US); Markus Landthaler, New York, NY (US); Gunter Meister, New York, NY (US); Sebastien Pfeffer, New York, NY (US)

Correspondence Address: HOFFMANN & BARON, LLP, 6900 JERICHO TURNPIKE, SYOSSET, NY, 11791, US

(21) Appl. No.: 10/845,057

(22) Filed: May 13, 2004

Related U.S. Application Data
(63) Continuation of application No. 10/778,908, filed on Feb. 13, 2004.

(51) Int. Cl. A61K 48/00; C07H 21/02

(52) U.S. Cl. 514/44; 536/23.1

(57) ABSTRACT

The invention relates to isolated anti-microRNA molecules. In another embodiment, the invention relates to an isolated microRNA molecule. In yet another embodiment, the invention provides a method for inhibiting microRNP activity in a cell.

This application is a continuing application of U.S. application Ser. No. 10/778,908 filed on Feb. 13, 2004. The specification of U.S. application Ser. No. 10/778,908 is hereby incorporated by reference in its entirety.

The invention claimed herein was made with the help of grant number 1 RO 1 GM068476-01 from NIH/NIGMS. The U.S. government has certain rights in the invention.

### BACKGROUND OF THE INVENTION

RNA silencing is a fundamental mechanism of gene regulation that uses double-stranded RNA (dsRNA) derived 21- to 28-nucleotide (nt) small RNAs to guide mRNA degradation, control mRNA translation or chromatin modification. Recently, several hundred novel genes were identified in plants and animals that encode transcripts that contain short dsRNA hairpins.

Defined 22-nt RNAs, referred to as microRNAs (miRNAs), are reported to be excised by dsRNA specific endonucleases from the hairpin precursors. The miRNAs are incorporated into ribonucleoprotein particles (miRNPs).

Plant miRNAs target mRNAs containing sequence segments with high complementarity for degradation or suppress translation of partially complementary mRNAs. Animal miRNAs appear to act predominantly as translational repressors. However, animal miRNAs have also been reported to guide RNA degradation. This indicates that animal miRNPs act like small interfering RNA (siRNA)-induced silencing complexes (RISCs).

Understanding the biological function of miRNAs requires knowledge of their mRNA targets. Bioinformatic approaches have been used to predict mRNA targets, among which transcription factors and proapoptotic genes were prominent candidates. Processes such as Notch signaling, cell proliferation, morphogenesis and axon guidance appear to be controlled by miRNA genes.

Therefore, there is a need for materials and methods that can help elucidate the function of known and future microRNAs. Due to the ability of microRNAs to induce RNA degradation or repress translation of mRNA which encode important proteins, there is also a need for novel compositions for inhibiting microRNA-indcued cleavage or repression of mRNAs.

### SUMMARY THE INVENTION

In one embodiment, the invention provides an isolated single stranded anti-microRNA molecule comprising a minimum of ten moieties and a maximum of fifty moieties on a molecular backbone, the molecular backbone comprising backbone units, each moiety comprising a base bonded to a backbone unit, each base forming a Watson-Crick base pair with a complementary base wherein at least ten contiguous bases have the same sequence as a sequence of bases in any one of the anti-microRNA molecules shown in Tables 1-4, except that up to thirty percent of the bases pairs may be wobble base pairs, and up to 10% of the contiguous bases may be additions, deletions, mismatches, or combinations thereof; no more than fifty percent of the contiguous moieties contain deoxyribonuleotide backbone units; the moiety in the molecule at the position corresponding to position 11 of the microRNA is non-complementary; and the molecule is capable of inhibiting microRNP activity.

In another embodiment, the invention provides a method for inhibiting microRNP activity in a cell, the microRNP comprising a microRNA molecule, the microRNA molecule comprising a sequences of bases complementary of the sequence of bases in a single stranded anti-microRNA molecule, the method comprising introducing into the cell the single-stranded anti-microRNA molecule comprising a sequence of a minimum of ten moieties and a maximum of fifty moieties on a molecular backbone, the molecular backbone comprising backbone units, each moiety comprising a base bonded to a backbone unit, each base forming a Watson-Crick base pair with a complementary base, wherein at least ten contiguous bases of the anti-microRNA molecule are complementary to the microRNA, except that up to thirty percent of the bases may be substituted by wobble base pairs, and up to ten percent of the at least ten moieties may be additions, deletions, mismatches, or combinations thereof; no more than fifty percent of the contiguous moieties contain deoxyribonuleotide backbone units; and the moiety in the molecule at the position corresponding to position 11 of the microRNA is non-complementary.

In another embodiment, the invention provides an isolated microRNA molecule comprising a minimum of ten moieties and a maximum of fifty moieties on a molecular backbone, the molecular backbone comprising backbone units, each moiety comprising a base bonded to a backbone unit, wherein at least ten contiguous bases have the same sequence as a sequence of bases in any one of the microRNA molecules shown in Table 2, except that up to thirty percent of the bases pairs may be wobble base pairs, and up to 10% of the contiguous bases may be additions, deletions, mismatches, or combinations thereof; and no more than fifty percent of the contiguous moieties contain deoxyribonuleotide backbone units.

In another embodiment, the invention provides an isolated microRNA molecule comprising a minimum of ten moieties and a maximum of fifty moieties on a molecular backbone, the molecular backbone comprising backbone units, each moiety comprising a base bonded to a backbone unit, wherein at least ten contiguous bases have any one of the microRNA sequences shown in Tables 1, 3 and 4, except that up to thirty percent of the bases pairs may be wobble base pairs, and up to 10% of the contiguous bases may be additions, deletions, mismatches, or combinations thereof; no more than fifty percent of the contiguous moieties contain deoxyribonuleotide backbone units; and is modified for increased nuclease resistance.

In yet another embodiment, the invention provides an isolated single stranded anti-microRNA molecule comprising a minimum of ten moieties and a maximum of fifty moieties on a molecular backbone, the molecular backbone comprising backbone units, each moiety comprising a base bonded to a backbone unit, each base forming a Watson-Crick base pair with a complementary base wherein at least ten contiguous bases have the same sequence as a sequence of bases in any one of the anti-microRNA molecules shown in Tables 1-4, except that up to thirty percent of the bases pairs may be wobble base pairs, and up to 10% of the contiguous bases may be additions, deletions, mismatches, or combinations thereof; no more than fifty percent of the contiguous moieties contain deoxyribonuleotide backbone units; and the molecule is capable of inhibiting microRNP activity.

In yet a further embodiment, the invention provides a method for inhibiting microRNP activity in a cell, the microRNP comprising a microRNA molecule, the microRNA molecule comprising a sequences of bases complementary of the sequence of bases in a single stranded anti-microRNA molecule, the method comprising introducing into the cell the single-stranded anti-microRNA molecule comprising a sequence of a minimum of ten moieties and a maximum of fifty moieties on a molecular backbone, the molecular backbone comprising backbone units, each moiety comprising a base bonded to a backbone unit, each base forming a Watson-Crick base pair with a complementary base, wherein at least ten contiguous bases of the anti-microRNA molecule are complementary to the microRNA, except that up to thirty percent of the bases may be substituted by wobble base pairs, and up to ten percent of the at least ten moieties may be additions, deletions, mismatches, or combinations thereof; and no more than fifty percent of the contiguous moieties contain deoxyribonuleotide backbone units.

### DESCRIPTION OF THE FIGURES

FIG. 1 shows the modified nucleotide units discussed in the specification. B denotes any one of the following nucleic acid bases: adenosine, cytidine, guanosine, thymine, or uridine.

FIG. 2. Antisense 2′-O-methyl oligoribonucleotide specifically inhibit miR-21 guided cleavage activity in HeLa cell S100 cytoplasmic extracts. The black bar to the left of the RNase T1 ladder represents the region of the target RNA complementary to miR-21. Oligonucleotides complementary to miR-21 were pre-incubated in S100 extracts prior to the addition of ³²P-cap-labelled cleavage substrate. Cleavage bands and T1 hydrolysis bands appear as doublets after a 1-nt slipping of the T7 RNA polymerase near the middle of the transcript indicated by the asterisk.

FIG. 3. Antisense 2′-O-methyl oligoribonucleotides interfere with endogenous miR-21 RNP cleavage in HeLa cells. HeLa cells were transfected with pHcRed and pEGFP or its derivatives, with or without inhibitory or control oligonucleotides. EGFP and HcRed protein fluorescence were excited and recorded individually by fluorescence microscopy 24 h after transfection. Co-expression of co-transfected reporter plasmids was documented by superimposing of the fluorescence images in the right panel.

### DETAILED DESCRIPTION OF THE INVENTION

The invention relates to an isolated single stranded anti-microRNA molecule. The molecule comprises a minimum number of ten moieties, preferably a minimum of thirteen, more preferably a minimum of fifteen, even more preferably a minimum of 18, and most preferably a minimum of 21 moieties.

The anti-microRNA molecule comprises a maximum number of fifty moieties, preferably a maximum of forty, more preferably a maximum of thirty, even more preferably a maximum of twenty-five, and most preferably a maximum of twenty-three moieties. A suitable range of minimum and maximum number of moieties may be obtained by combining any of the above minima with any of the above maxima.

Each moiety comprises a base bonded to a backbone unit. In this specification, a base refers to any one of the nucleic acid bases present in DNA or RNA. The base can be a purine or pyrimidine. Examples of purine bases include adenine (A) and guanine (G). Examples of pyrimidine bases include thymine (T), cytosine (C) and uracil (U). Each base of the moiety forms a Watson-Crick base pair with a complementary base.

Watson-Crick base pairs as used herein refers to the hydrogen bonding interaction between, for example, the following bases: adenine and thymine (A=T); adenine and uracil (A=U); and cytosine and guanine (C=G). The adenine can be replaced with 2,6-diaminopurine without compromising base-pairing.

The backbone unit may be any molecular unit that is able stably to bind to a base and to form an oligomeric chain. Suitable backbone units are well known to those in the art.

For example, suitable backbone units include sugar-phosphate groups, such as the sugar-phosphate groups present in ribonucleotides, deoxyribonucleotides, phosphorothioate deoxyribose groups, N′3-N′5 phosphoroamidate deoxyribose groups, 2′O-alkyl-ribose phosphate groups, 2′-O-alkyl-alkoxy ribose phosphate groups, ribose phosphate group containing a methylene bridge, 2′-Fluororibose phosphate groups, morpholino phosphoroamidate groups, cyclohexene groups, tricyclo phosphate groups, and amino acid molecules.

In one embodiment, the anti-microRNA molecule comprises at least one moiety which is a ribonucleotide moiety or a deoxyribonucleotide moiety.

In another embodiment, the anti-microRNA molecule comprises at least one moiety which confers increased nuclease resistance. The nuclease can be an exonuclease, an endonuclease, or both. The exonuclease can be a 3′→5′ exonuclease or a 5′→3′ exonuclease. Examples of 3′→5′ human exonuclease include PNPT1, Werner syndrome helicase, RRP40, RRP41, RRP42, RRP45, and RRP46. Examples of 5′→3′ exonuclease include XRN2, and FEN1. Examples of endonucleases include Dicer, Drosha, RNase4, Ribonuclease P, Ribonuclease H1, DHP1, ERCC-1 and OGG1. Examples of nucleases which function as both an exonuclease and an endonuclease include APE1 and EXO1.

An anti-microRNA molecule comprising at least one moiety which confers increased nuclease resistance means a sequence of moieties wherein at least one moiety is not recognized by a nuclease. Therefore, the nuclease resistance of the molecule is increased compared to a sequence containing only unmodified ribonucleotide, unmodified deoxyribonucleotide or both. Such modified moieties are well known in the art, and were- reviewed, for example, by Kurreck, Eur. J. Biochem. 270, 1628-1644 (2003).

A modified moiety can occur at any position in the anti-microRNA molecule. For example, to protect the anti-microRNA molecule against 3′→5′ exonucleases, the molecule can have at least one modified moiety at the 3′ end of the molecule and preferably at least two modified moieties at the 3′ end. If it is desirable to protect the molecule against 5′→3′ exonuclease, the anti-microRNA molecule can have at least one modified moiety and preferably at least two modified moieties at the 5′ end of the molecule. The anti-microRNA molecule can also have at least one and preferably at least two modified moieties between the 5′ and 3′ end of the molecule to increase resistance of the molecule to endonucleases. In one embodiment, all of the moieties are nuclease resistant.

In another embodiment, the anti-microRNA molecule comprises at least one modified deoxyribonucleotide moiety. Suitable modified deoxyribonucleotide moieties are known in the art.

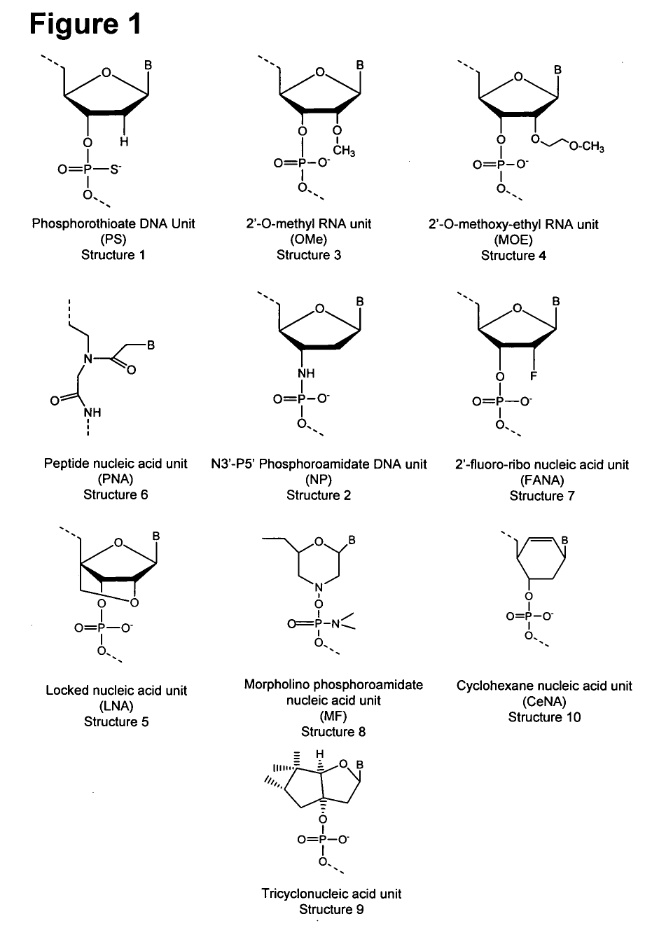

A suitable example of a modified deoxyribonucleotide moiety is a phosphorothioate deoxyribonucleotide moiety. See structure 1 in FIG. 1. An anti-microRNA molecule comprising more than one phosphorothioate deoxyribonucleotide moiety is referred to as phosphorothioate (PS) DNA. See, for example, Eckstein, Antisense Nucleic Acids Drug Dev. 10, 117-121 (2000).

Another suitable example of a modified deoxyribonucleotide moiety is an N′3-N′5 phosphoroamidate deoxyribonucleotide moiety. See structure 2 in FIG. 1. An oligonucleotide molecule comprising more than one phosphoroamidate deoxyribonucleotide moiety is referred to as phosphoroamidate (NP) DNA. See, for example, Gryaznov et al., J. Am. Chem. Soc. 116, 3143-3144 (1994).

In another embodiment, the molecule comprises at least one modified ribonucleotide moiety. Suitable modified ribonucleotide moieties are known in the art.

A suitable example of a modified ribonucleotide moiety is a ribonucleotide moiety that is substituted at the 2′ position. The substituents at the 2′ position may, for example, be a C₁ to C₄ alkyl group. The C₁ to C₄ alkyl group may be saturated or unsaturated, and unbranched or branched. Some examples of C₁ to C₄ alkyl groups include ethyl, isopropyl, and allyl. The preferred C₁ to C₄ alkyl group is methyl. See structure 3 in FIG. 1. An oligoribonucleotide molecule comprising more than one ribonucleotide moeity that is substituted at the 2′ position with a C₁ to C₄ alkyl group is referred to as a 2′-O—(C₁-C₄ alkyl) RNA, e.g., 2′-O-methyl RNA (OMe RNA).

Another suitable example of a substituent at the 2′ position of a modified ribonucleotide moiety is a C₁ to C₄ alkoxy-C₁ to C₄ alkyl group. The C₁ to C₄ alkoxy (alkyloxy) and C₁ to C₄ alkyl group may comprise any of the alkyl groups described above. The preferred C₁ to C₄ alkoxy-C₁ to C₄ alkyl group is methoxyethyl. See structure 4 in FIG. 1. An ogligonucleotide molecule comprising more than one ribonucleotide moiety that is substituted at the 2′ position with a C1 to C₄ alkoxy-C₁ to C₄ alkyl group is referred to as a 2′-O—(C₁ to C₄ alkoxy-C₁ to C₄ alkyl) RNA, e.g., 2′-O-methoxyethyl RNA (MOE RNA).

Another suitable example of a modified ribonucleotide moiety is a ribonucleotide that has a methylene bridge between the 2′-oxygen atom and the 4′-carbon atom. See structure 5 in FIG. 1. An oligoribonucleotide molecule comprising more than one ribonucleotide moiety that has a methylene bridge between the 2′-oxygen atom and the 4′-carbon atom is referred to as locked nucleic acid (LNA). See, for example, Kurreck et al., Nucleic Acids Res. 30, 1911- 1918 (2002); Elayadi et al., Curr. Opinion Invest. Drugs 2, 558-561 (2001); Ørum et al., Curr. Opinion Mol. Ther. 3, 239-243 (2001); Koshkin et al., Tetrahedron 54, 3607-3630 (1998); Obika et al., Tetrahedron Lett. 39, 5401-5404 (1998). Locked nucleic acids are commercially available from Proligo (Paris, France and Boulder, Col., USA).

Another suitable example of a modified ribonucleotide moiety is a ribonucleotide that is substituted at the 2′ position with fluoro group. A modified ribonucleotide moiety having a fluoro group at the 2′ position is a 2′-fluororibonucleotide moiety. Such moieties are known in the art. Molecules comprising more than one 2′-fluororibonucleotide moiety are referred to herein as 2′-fluororibo nucleic acids (FANA). See structure 7 in FIG. 1. Damha et al., J. Am. Chem. Soc. 120, 12976-12977 (1998).

In another embodiment, the anti-microRNA molecule comprises at least one base bonded to an amino acid residue. Moieties that have at least one base bonded to an amino acid residue will be referred to herein as peptide nucleic acid (PNA) moieties. Such moieties are nuclease resistance, and are known in the art. Molecules having more than one PNA moiety are referred to as peptide nucleic acids. See structure 6 in FIG. 1. Nielson, Methods Enzymol. 313, 156-164 (1999); Elayadi, et al, id.; Braasch et al., Biochemistry 41, 4503-4509 (2002), Nielsen et al., Science 254, 1497-1500 (1991).

The amino acids can be any amino acid, including natural or non-natural amino acids. Naturally occurring amino acids include, for example, the twenty most common amino acids normally found in proteins, i.e., alanine (Ala), arginine (Arg), asparagine (Asn), aspartic acid (Asp), cysteine (Cys), glutamine (Glu), glutamic acid (Glu), glycine (Gly), histidine (His), isoleucine (Ileu), leucine (Leu), lysine (Lys), methionine (Met), phenylalanine (Phe), proline (Pro), serine (Ser), threonine (Thr), tryptophan, (Trp), tyrosine (Tyr), and valine (Val).

The non-natural amino acids may, for example, comprise alkyl, aryl, or alkylaryl groups. Some examples of alkyl amino acids include α-aminobutyric acid, β-aminobutyric acid, γ-aminobutyric acid, δ-aminovaleric acid, and ε-aminocaproic acid. Some examples of aryl amino acids include ortho-, meta, and para-aminobenzoic acid. Some examples of alkylaryl amino acids include ortho-, meta-, and para-aminophenylacetic acid, and γ-phenyl-β-aminobutyric acid.

Non-naturally occurring amino acids also include derivatives of naturally occurring amino acids. The derivative of a naturally occurring amino acid may, for example, include the addition or one or more chemical groups to the naturally occurring amino acid.

For example, one or more chemical groups can be added to one or more of the 2′, 3′, 4′, 5′, or 6′ position of the aromatic ring of a phenylalanine or tyrosine residue, or the 4′, 5′, 6′, or 7′ position of the benzo ring of a tryptophan residue. The group can be any chemical group that can be added to an aromatic ring. Some examples of such groups include hydroxyl, C₁-C₄ alkoxy, amino, methylamino, dimethylamino, nitro, halo (i.e., fluoro, chloro, bromo, or iodo), or branched or unbranched C₁-C₄ alkyl, such as methyl, ethyl, n-propyl, isopropyl, butyl, isobutyl, or t-butyl.

Furthermore, other examples of non-naturally occurring amino acids which are derivatives of naturally occurring amino acids include norvaline (Nva), norleucine (Nle), and hydroxyproline (Hyp).

The amino acids can be identical or different from one another. Bases are attached to the amino acid unit by molecular linkages. Examples of linkages are methylene carbonyl, ethylene carbonyl and ethyl linkages. (Nielsen et al., Peptide Nucleic Acids—Protocols and Applications, Horizon Scientific Press, pages 1-19; Nielsen et al., Science 254: 1497-1500.)

One example of a PNA moiety is N-(2-aminoethyl)-glycine. Further examples of PNA moieties include cyclohexyl PNA, retro-inverso, phosphone, propionyl and aminoproline PNA.

PNA can be chemically synthesized by methods known in the art, e.g. by modified Fmoc or tBoc peptide synthesis protocols. The PNA has many desirable properties, including high melting temperatures (Tm), high base-pairing specificity with nucleic acid and an uncharged molecular backbone. Additionally, the PNA does not confer RNase H sensitivity on the target RNA, and generally has good metabolic stability.

Peptide nucleic acids are also commercially available from Applied Biosystems (Foster City, Calif., USA).

In another embodiment, the anti-microRNA molecule comprises at least one morpholino phosphoroamidate nucleotide moiety. A morpholino phosphoroamidate nucleotide moiety is a modified moiety which is nuclease resistant. Such moieties are known in the art. Molecules comprising more than one morpholino phosphoroamidate nucleotide moiety are referred to as morpholino (MF) nucleic acids. See structure 8 in FIG. 1. Heasman, Dev. Biol. 243, 209-214 (2002). Morpholono oligonucleotides are commercially available from Gene Tools LLC (Corvallis, Oreg., USA).

In another embodiment, the anti-microRNA molecule comprises at least one cyclohexene nucleotide moiety. A cyclohexene nucleotide moiety is a modified moiety which is nuclease resistant. Such moieties are known in the art. Molecules comprising more than one cyclohexene nucleotide moiety are referred to as cyclohexene nucleic acids (CeNA). See structure 10 in FIG. 1. Wang et al., J. Am. Chem. Soc. 122, 8595-8602 (2000), Verbeure et al., Nucleic Acids Res. 29, 4941-4947 (2001).

In another embodiment, the anti-microRNA molecule comprises at least one tricyclo nucleotide moiety. A tricyclo nucleotide moiety is a modified moiety which is nuclease resistant. Such moieties are known in the art. Steffens et al., J. Am. Chem. Soc. 119, 11548-11549 (1997), Renneberg et al., J. Am. Chem. Soc. 124, 5993-6002 (2002). Molecules comprising more than one tricyclo nucleotide moiety are referred to as tricyclo nucleic acids (tcDNA). See structure 9 in FIG. 1.

In another embodiment, to increase nuclease resistance of the anti-microRNA molecules of the present invention to exonucleases, inverted nucleotide caps can be attached to the 5′ end, the 3′ end, or both ends of the molecule. An inverted nucleotide cap refers to a 3′→5′ sequence of nucleic acids attached to the anti-microRNA molecule at the 5′ and/or the 3′ end. There is no limit to the maximum number of nucleotides in the inverted cap just as long as it does not interfere with binding of the anti-microRNA molecule to its target microRNA. Any nucleotide can be used in the inverted nucleotide cap. Typically, the inverted nucleotide cap is one nucleotide in length. The nucleotide for the inverted cap is generally thymine, but can be any nucleotide such as adenine, guanine, uracil, or cytosine.

Alternatively, an ethylene glycol compound and/or amino linkers can be attached to the either or both ends of the anti-microRNA molecule. Amino linkers can also be used to increase nuclease resistance of the anti-microRNA molecules to endonucleases. The table below lists some examples of amino linkers. The below listed amino linker are commercially available from TriLink Biotechnologies, San Diego, Calif.

2′-Deoxycytidine-5-C6 Amino Linker (3′ Terminus)
2′-Deoxycytidine-5-C6 Amino Linker (5′ or Internal)
3′ C3 Amino Linker
3′ C6 Amino Linker
3′ C7 Amino Linker
5′ C12 Amino Linker
5′ C3 Amino Linker
5′ C6 Amino Linker
C7 Internal Amino Linker
Thymidine-5-C2 Amino Linker (5′ or Internal)
Thymidine-5-C6 Amino Linker (3′ Terminus)
Thymidine-5-C6 Amino Linker (Internal)

Chimeric anti-microRNA molecules containing a mixture of any of the moieties mentioned above are also known, and may be made by methods known, in the art. See, for example, references cited above, and Wang et al, Proc. Natl. Acad. Sci. USA 96, 13989-13994 (1999), Liang et al., Eur. J. Biochem. 269, 5753-5758 (2002), Lok et al., Biochemistry 41, 3457-3467 (2002), and Damha et al., J. Am. Chem. Soc. 120, 12976-12977 (2002).

The molecules of the invention comprise at least ten contiguous, preferably at least thirteen contiguous, more preferably at least fifteen contiguous, and even more preferably at least twenty contiguous bases that have the same sequence as a sequence of bases in any one of the anti-microRNA molecules shown in Tables 1-4. The anti-microRNA molecules optimally comprise the entire sequence of any one of the anti-microRNA molecule sequences shown in Tables 1-4.

For the contiguous bases mentioned above, up to thirty percent of the base pairs may be substituted by wobble base pairs. As used herein, wobble base pairs refers to either: i) substitution of a cytosine with a uracil, or 2) the substitution of a adenine with a guanine, in the sequence of the anti-microRNA molecule. These wobble base pairs are generally referred to as UG or GU wobbles. Below is a table showing the number of contiguous bases and the maximum number of wobble base pairs in the anti-microRNA molecule:

Table for Number of Wobble Bases

| | | | | | | | | | | | | | | |
|---|---|---|---|---|---|---|---|---|---|---|---|---|---|---|
| No. | 10 | 11 | 12 | 13 | 14 | 15 | 16 | 17 | 18 | 19 | 20 | 21 | 22 | 23 |
| of | | | | | | | | | | | | | | |
| Con- | | | | | | | | | | | | | | |
| tig- | | | | | | | | | | | | | | |
| uous | | | | | | | | | | | | | | |
| Bases | | | | | | | | | | | | | | |
| Max. | 3 | 3 | 3 | 3 | 4 | 4 | 4 | 5 | 5 | 5 | 6 | 6 | 6 | 6 |
| No. | | | | | | | | | | | | | | |
| of | | | | | | | | | | | | | | |
| Wob- | | | | | | | | | | | | | | |
| ble | | | | | | | | | | | | | | |
| Base | | | | | | | | | | | | | | |
| Pairs | | | | | | | | | | | | | | |

Further, up to ten percent, and preferably up to five percent of the contiguous bases can be additions, deletions, mismatches or combinations thereof. Additions refer to the insertion in the contiguous sequence of any moiety described above comprising any one of the bases described above. Deletions refer to the removal of any moiety present in the contiguous sequence. Mismatches refer to the substitution of one of the moieties comprising a base in the contiguous sequence with any of the above described moieties comprising a different base.

The additions, deletions or mismatches can occur anywhere in the contiguous sequence, for example, at either end of the contiguous sequence or within the contiguous sequence of the anti-microRNA molecule. If the contiguous sequence is relatively short, such as from about ten to about 15 moieties in length, preferably the additions, deletions or mismatches occur at the end of the contiguous sequence. If the contiguous sequence is relatively long, such as a minimum of sixteen contiguous sequences, then the additions, deletions, or mismatches can occur anywhere in the contiguous sequence. Below is a table showing the number of contiguous bases and the maximum number of additions, deletions, mismatches or combinations thereof:

Table for Up to 10%

| | | | | | | | | | | | | | | |
|---|---|---|---|---|---|---|---|---|---|---|---|---|---|---|
| No. of | 10 | 11 | 12 | 13 | 14 | 15 | 16 | 17 | 18 | 19 | 20 | 21 | 22 | 23 |
| Contiguous | | | | | | | | | | | | | | |
| Bases | | | | | | | | | | | | | | |
| Max. No. of | 1 | 1 | 1 | 1 | 1 | 1 | 1 | 1 | 1 | 1 | 2 | 2 | 2 | 2 |
| Additions, | | | | | | | | | | | | | | |
| Deletions | | | | | | | | | | | | | | |
| and/or | | | | | | | | | | | | | | |
| Mismatches | | | | | | | | | | | | | | |

Table for Up to 5%

| | | | | | | | | | | | | | | |
|---|---|---|---|---|---|---|---|---|---|---|---|---|---|---|
| No. of | 10 | 11 | 12 | 13 | 14 | 15 | 16 | 17 | 18 | 19 | 20 | 21 | 22 | 23 |
| Contiguous | | | | | | | | | | | | | | |
| Bases | | | | | | | | | | | | | | |
| Max. No. of | 0 | 0 | 0 | 0 | 0 | 0 | 0 | 0 | 0 | 0 | 1 | 1 | 1 | 1 |
| Additions, | | | | | | | | | | | | | | |
| Deletions | | | | | | | | | | | | | | |
| and/or | | | | | | | | | | | | | | |
| Mismatches | | | | | | | | | | | | | | |

Furthermore, no more than fifty percent, and preferably no more than thirty percent, of the contiguous moieties contain deoxyribonucleotide backbone units. Below is a table showing the number of contiguous bases and the maximum number of deoxyribonucleotide backbone units:

Table for Fifty Percent Deoxyribonucleotide Backbone Units

| | | | | | | | | | | | | | | |
|---|---|---|---|---|---|---|---|---|---|---|---|---|---|---|
| No. of | 10 | 11 | 12 | 13 | 14 | 15 | 16 | 17 | 18 | 19 | 20 | 21 | 22 | 23 |
| Contiguous | | | | | | | | | | | | | | |
| Bases | | | | | | | | | | | | | | |
| Max. No. of | 5 | 5 | 6 | 6 | 7 | 7 | 8 | 8 | 9 | 9 | 10 | 10 | 11 | 11 |
| Deoxyribo- | | | | | | | | | | | | | | |
| nucleotide | | | | | | | | | | | | | | |
| Backbone | | | | | | | | | | | | | | |
| Units | | | | | | | | | | | | | | |

Table for Thirty Percent Deoxyribonucleotide Backbone Units

| | | | | | | | | | | | | | | |
|---|---|---|---|---|---|---|---|---|---|---|---|---|---|---|
| No. of | 10 | 11 | 12 | 13 | 14 | 15 | 16 | 17 | 18 | 19 | 20 | 21 | 22 | 23 |
| Contiguous | | | | | | | | | | | | | | |
| Bases | | | | | | | | | | | | | | |
| Max. No. of | 3 | 3 | 3 | 3 | 4 | 4 | 4 | 5 | 5 | 5 | 6 | 6 | 6 | 6 |
| Deoxyribo- | | | | | | | | | | | | | | |
| nucleotide | | | | | | | | | | | | | | |
| Backbone | | | | | | | | | | | | | | |
| Units | | | | | | | | | | | | | | |

The moiety in the anti-RNA molecule at the position corresponding to position 11 of the microRNA is optionally non-complementary to a microRNA. The moiety in the anti-microRNA molecule corresponding to position 11 of the microRNA can be rendered non-complementary by an addition, deletion or mismatch as described above.

In another embodiment, if the anti-microRNA molecule comprises only unmodified moieties, then the anti-microRNA molecules comprises at least one base, in the at least ten contiguous bases, which is non-complementary to the microRNA and/or comprises an inverted nucleotide cap, ethylene glycol compound or an amino linker.

In yet another embodiment, if the at least ten contiguous bases in an anti-microRNA molecule is perfectly (i.e., 100%) complementary to ten contiguous bases in a microRNA, then the anti-microRNA molecule contains at least one modified moiety in the at least ten contiguous bases and/or comprises an inverted nucleotide cap, ethylene glycol compound or an amino linker.

As stated above, the maximum length of the anti-microRNA molecule is 50 moieties. Any number of moieties having any base sequence can be added to the contiguous base sequence. The additional moieties can be added to the 5′ end, the 3′ end, or to both ends of the contiguous sequence.

MicroRNA molecules are derived from genomic loci and are produced from specific microRNA genes. Mature microRNA molecules are processed from precursor transcripts that form local hairpin structures. The hairpin structures are typically cleaved by an enzyme known as Dicer, which generates one microRNA duplex. See Bartel, Cell 116, 281-297 (2004) for a review on microRNA molecules. The article by Bartel is hereby incorporated by reference.

Each strand of a microRNA is packaged in a microRNA ribonucleoprotein complex (microRNP). A microRNP in, for example, humans, also includes the proteins eIF2C2, the helicase Gemin3, and Gemin 4.

The sequence of bases in the anti-microRNA molecules of the present invention can be derived from a microRNA from any species e.g. such as a fly (e.g., Drosophila melanogaster), a worm (e.g., C. elegans). Preferably the sequence of bases is found in mammals, especially humans (H. sapiens), mice (e.g., M. musculus), and rats (R. norvegicus).

The anti-microRNA molecule is preferably isolated, which means that it is essentially free of other nucleic acids. Essentially free from other nucleic acids means that it is at least 90%, preferably at least 95% and, more preferably, at least 98% free of other nucleic acids.

Preferably, the molecule is essentially pure, which means that the molecules is free not only of other nucleic acids, but also of other materials used in the synthesis of the molecule, such as, for example, enzymes used in the synthesis of the molecule. The molecule is at least 90% free, preferably at least 95% free and, more preferably, at least 98% free of such materials.

The anti-microRNA molecules of the present invention are capable of inhibiting microRNP activity, preferable in a cell. Inhibiting microRNP activity refers to the inhibition of cleavage of the microRNA's target sequence or the repression of translation of the microRNA's target sequence. The method comprises introducing into the cell a single-stranded microRNA molecule.

Any anti-microRNA molecule can be used in the methods of the present invention, as long as the anti-microRNA is complementary, subject to the restrictions described above, to the microRNA present in the microRNP. Such anti-microRNAs include, for example, the anti-microRNA molecules mentioned above (see Table 1-4), and the anti-microRNAs molecules described in international PCT application No. WO 03/029459 A2, the sequences of which are incorporated herein by reference.

The invention also includes any one of the microRNA molecules having the sequences as shown in Table 2. The novel microRNA molecules in Table 2 may optionally be modified as described above for anti-microRNA molecules. The other microRNA molecules in Tables 1, 3 and 4 are modified for increased nuclease resistance as described above for anti-microRNA molecules.

Utility

The anti-microRNA molecules and the microRNA molecules of the present invention have numerous in vivo, in vitro, and ex vivo applications.

For example, the anti-microRNA molecules and microRNA of the present invention may be used as a modulator of the expression of genes which are at least partially complementary to the anti-microRNA molecules and microRNA. For example, if a particular microRNA is beneficial for the survival of a cell, an appropriate isolated microRNA of the present invention may be introduced into the cell to promote survival. Alternatively, if a particular microRNA is harmful (e.g., induces apoptosis, induces cancer, etc.), an appropriate anti-microRNA molecule can be introduced into the cell in order to inhibit the activity of the microRNA and reduce the harm.

In addition, anti-microRNA molecules and/or microRNAs of the present invention can be introduced into a cell to study the function of the microRNA. Any of the anti-microRNA molecules and/or microRNAs listed above can be introduced into a cell for studying their function. For example, a microRNA in a cell can be inhibited with a suitable anti-microRNA molecule. The function of the microRNA can be inferred by observing changes associated with inhibition of the microRNA in the cell in order to inhibit the activity of the microRNA and reduce the harm.

The cell can be any cell which expresses microRNA molecules, including the microRNA molecules listed herein. Alternatively, the cell can be any cell transfected with an expression vector containing the nucleotide sequence of a microRNA.

Examples of cells include, but are not limited to, endothelial cells, epithelial cells, leukocytes (e.g., T cells, B cells, neutrophils, macrophages, eosinophils, basophils, dendritic cells, natural killer cells and monocytes), stem cells, hemopoietic cells, embryonic cells, cancer cells.

The anti-microRNA molecules or microRNAs can be introduced into a cell by any method known to those skilled in the art. Useful delivery systems, include for example, liposomes and charged lipids. Liposomes typically encapsulate oligonucleotide molecules within their aqueous center. Charged lipids generally form lipid- oligonucleotide molecule complexes as a result of opposing charges.

These liposomes-oligonucleotide molecule complexes or lipid-oligonucleotide molecule complexes are usually internalized by endocytosis. The liposomes or charged lipids generally comprise helper lipids which disrupt the endosomal membrane and release the oligonucleotide molecules.

Other methods for introducing an anti-microRNA molecule or a microRNA into a cell include use of delivery vehicles, such as dendrimers, biodegradable polymers, polymers of amino acids, polymers of sugars, and oligonucleotide-binding nanoparticles. In addition, pluoronic gel as a depot reservoir can be used to deliver the anti-microRNA oligonucleotide molecules over a prolonged period. The above methods are described in, for example, Hughes et al., Drug Discovery Today 6, 303-315 (2001); Liang et al. Eur. J. Biochem. 269 5753-5758 (2002); and Becker et al., In Antisense Technology in the Central Nervous System (Leslie, R. A., Hunter, A. J. & Robertson, H. A., eds), pp. 147-157, Oxford University Press.

Targeting of an anti-microRNA molecule or a microRNA to a particular cell can be performed by any method known to those skilled in the art. For example, the anti-microRNA molecule or microRNA can be conjugated to an antibody or ligand specifically recognized by receptors on the cell.

The sequences of microRNA and anti-microRNA molecules are shown in Tables 1-4 below. Human sequences are indicated with the prefix “hsa.” Mouse sequences are indicated with the prefix “mmu.” Rat sequences are indicated with the prefix “rno.” C. elegan sequences are indicated with the prefix “cel.” Drosophila sequences are indicated with the prefix “dme.”

TABLE 1

Human, Mouse and Rat microRNA and anti-microRNA
sequences.

| | |
|---|---|
| microRNA sequence | Anti-microRNA molecule |

| | | |
|---|---|---|
| microRNA name | (5′ to 3′) | sequence (5′ to 3′) |

| | | |
|---|---|---|
| hsa-miR-100 | AACCCGUAGAUCCGAACUUGUG | CACAAGUUCGGAUCUACGGGUU |
| hsa-miR-103 | AGCAGCAUUGUACAGGGCUAUG | CAUAGCCCUGUACAAUGCUGCU |
| hsa-miR-105-5p | UCAAAUGCUCAGACUCCUGUGG | CCACAGGAGUCUGAGCAUUUGA |
| hsa-miR-106a | AAAAGUGCUUACAGUGCAGGUA | UACCUGCACUGUAAGCACUUUU |
| hsa-miR-106b | UAAAGUGCUGACAGUGCAGAUA | UAUCUGCACUGUCAGCACUUUA |
| hsa-miR-107 | AGCAGCAUUGUACAGGGCUAUC | GAUAGCCCUGUACAAUGCUGCU |
| hsa-miR-10b | UACCCUGUAGAACCGAAUUUGU | ACAAAUUCGGUUCUACAGGGUA |
| hsa-miR-128b | UCACAGUGAACCGGUCUCUUUC | GAAAGAGACCGGUUCACUGUGA |
| hsa-miR-130b | CAGUGCAAUGAUGAAAGGGCAU | AUGCCCUUUCAUCAUUGCACUG |
| hsa-miR-140-3p | UACCACAGGGUAGAACCACGGA | UCCGUGGUUCUACCCUGUGGUA |
| hsa-miR-142-5p | CCCAUAAAGUAGAAAGCACUAC | GUAGUGCUUUCUACUUUAUGGG |
| hsa-miR-151-5p | UCGAGGAGCUCACAGUCUAGUA | UACUAGACUGUGAGCUCCUCGA |
| hsa-miR-155 | UUAAUGCUAAUCGUGAUAGGGG | CCCCUAUCACGAUUAGCAUUAA |
| hsa-miR-181a | AACAUUCAACGCUGUCGGUGAG | CUCACCGACAGCGUUGAAUGUU |
| hsa-miR-181b | AACAUUCAUUGCUGUCGGUGGG | CCCACCGACAGCAAUGAAUGUU |
| hsa-miR-181c | AACAUUCAACCUGUCGGUGAGU | ACUCACCGACAGGUUGAAUGUU |
| hsa-miR-182 | UUUGGCAAUGGUAGAACUCACA | UGUGAGUUCUACCAUUGCCAAA |
| hsa-miR-183 | UAUGGCACUGGUAGAAUUCACU | AGUGAAUUCUACCAGUGCCAUA |
| hsa-miR-184 | UGGACGGAGAACUGAUAAGGGU | ACCCUUAUCAGUUCUCCGUCCA |
| hsa-miR-185 | UGGAGAGAAAGGCAGUUCCUGA | UCAGGAACUGCCUUUCUCUCCA |
| hsa-miR-186 | CAAAGAAUUCUCCUUUUGGGCU | AGCCCAAAAGGAGAAUUCUUUG |
| hsa-miR-187 | UCGUGUCUUGUGUUGCAGCCGG | CCGGCUGCAACACAAGACACGA |
| hsa-miR-188-3p | CUCCCACAUGCAGGGUUUGCAG | CUGCAAACCCUGCAUGUGGGAG |
| hsa-miR-188-5p | CAUCCCUUGCAUGGUGGAGGGU | ACCCUCCACCAUGCAAGGGAUG |
| hsa-miR-189 | GUGCCUACUGAGCUGAUAUCAG | CUGAUAUCAGCUCAGUAGGCAC |
| hsa-miR-190 | UGAUAUGUUUGAUAUAUUAGGU | ACCUAAUAUAUCAAACAUAUCA |
| hsa-miR-191 | CAACGGAAUCCCAAAAGCAGCU | AGCUGCUUUUGGGAUUCCGUUG |
| hsa-miR-192 | CUGACCUAUGAAUUGACAGCCA | UGGCUGUCAAUUCAUAGGUCAG |
| hsa-miR-193-3p | AACUGGCCUACAAAGUCCCAGU | ACUGGGACUUUGUAGGCCAGUU |
| hsa-miR-193-5p | UGGGUCUUUGCGGGCAAGAUGA | UCAUCUUGCCCGCAAAGACCCA |
| hsa-miR-194 | UGUAACAGCAACUCCAUGUGGA | UCCACAUGGAGUUGCUGUUACA |
| hsa-miR-195 | UAGCAGCACAGAAAUAUUGGCA | UGCCAAUAUUUCUGUGCUGCUA |
| hsa-miR-196 | UAGGUAGUUUCAUGUUGUUGGG | CCCAACAACAUGAAACUACCUA |
| hsa-miR-197 | UUCACCACCUUCUCCACCCAGC | GCUGGGUGGAGAAGGUGGUGAA |
| hsa-miR-198 | GGUCCAGAGGGGAGAUAGGUUC | GAACCUAUCUCCCCUCUGGACC |
| hsa-miR-199a-3p | ACAGUAGUCUGCACAUUGGUUA | UAACCAAUGUGCAGACUACUGU |
| hsa-miR-199a-5p | CCCAGUGUUCAGACUACCUGUU | AACAGGUAGUCUGAACACUGGG |
| hsa-miR-199b | CCCAGUGUUAGACUAUCUGUUU | AACAGAUAGUCUAAACACUGGG |
| hsa-miR-200a | UAACACUGUCUGGUAACGAUGU | ACAUCGUUACCAGACAGUGUUA |
| hsa-miR-200b | CUCUAAUACUGCCUGGUAAUGA | UCAUUACCAGGCAGUAUUAGAG |
| hsa-miR-200c | AAUACUGCCGGGUAAUGAUGGA | UCCAUCAUUACCCGGCAGUAUU |
| hsa-miR-203 | GUGAAAUGUUUAGGACCACUAG | CUAGUGGUCCUAAACAUUUCAC |
| hsa-miR-204 | UUCCCUUUGUCAUCCUAUGCCU | AGGCAUAGGAUGACAAAGGGAA |
| hsa-miR-205 | UCCUUCAUUCCACCGGAGUCUG | CAGACUCCGGUGGAAUGAAGGA |
| hsa-miR-206 | UGGAAUGUAAGGAAGUGUGUGG | CCACACACUUCCUUACAUUCCA |
| hsa-miR-208 | AUAAGACGAGCAAAAAGCUUGU | ACAAGCUUUUUGCUCGUCUUAU |
| hsa-miR-210 | CUGUGCGUGUGACAGCGGCUGA | UCAGCCGCUGUCACACGCACAG |
| hsa-miR-211 | UUCCCUUUGUCAUCCUUCGCCU | AGGCGAAGGAUGACAAAGGGAA |
| hsa-miR-212 | UAACAGUCUCCAGUCACGGCCA | UGGCCGUGACUGGAGACUGUUA |
| hsa-miR-213 | ACCAUCGACCGUUGAUUGUACC | GGUACAAUCAACGGUCGAUGGU |
| hsa-miR-214 | ACAGCAGGCACAGACAGGCAGU | ACUGCCUGUCUGUGCCUGCUGU |
| hsa-miR-215 | AUGACCUAUGAAUUGACAGACA | UGUCUGUCAAUUCAUAGGUCAU |
| hsa-miR-216 | UAAUCUCAGCUGGCAACUGUGA | UCACAGUUGCCAGCUGAGAUUA |
| hsa-miR-217 | UACUGCAUCAGGAACUGAUUGG | CCAAUCAGUUCCUGAUGCAGUA |
| hsa-miR-218 | UUGUGCUUGAUCUAACCAUGUG | CACAUGGUUAGAUCAAGCACAA |
| hsa-miR-219 | UGAUUGUCCAAACGCAAUUCUU | AAGAAUUGCGUUUGGACAAUCA |
| hsa-miR-220 | CCACACCGUAUCUGACACUUUG | CAAAGUGUCAGAUACGGUGUGG |
| hsa-miR-221 | AGCUACAUUGUCUGCUGGGUUU | AAACCCAGCAGACAAUGUAGCU |
| hsa-miR-222 | AGCUACAUCUGGCUACUGGGUC | GACCCAGUAGCCAGAUGUAGCU |
| hsa-miR-223 | UGUCAGUUUGUCAAAUACCCCA | UGGGGUAUUUGACAAACUGACA |
| hsa-miR-224 | CAAGUCACUAGUGGUUCCGUUU | AAACGGAACCACUAGUGACUUG |
| hsa-miR-28-5p | AAGGAGCUCACAGUCUAUUGAG | CUCAAUAGACUGUGAGCUCCUU |
| hsa-miR-290 | CUCAAACUGUGGGGGCACUUUC | GAAAGUGCCCCCACAGUUUGAG |
| hsa-miR-296 | AGGGCCCCCCCUCAAUCCUGUU | AACAGGAUUGAGGGGGGGCCCU |
| hsa-miR-299 | UGGUUUACCGUCCCACAUACAU | AUGUAUGUGGGACGGUAAACCA |
| hsa-miR-301 | CAGUGCAAUAGUAUUGUCAAAG | CUUUGACAAUACUAUUGCACUG |
| hsa-miR-302 | UAAGUGCUUCCAUGUUUUGGUG | CACCAAAACAUGGAAGCACUUA |
| hsa-miR-30e | UGUAAACAUCCUUGACUGGAAG | CUUCCAGUCAAGGAUGUUUACA |
| hsa-miR-320 | AAAAGCUGGGUUGAGAGGGCGA | UCGCCCUCUCAACCCAGCUUUU |
| hsa-miR-321 | UAAGCCAGGGAUUGUGGGUUCG | CGAACCCACAAUCCCUGGCUUA |
| hsa-miR-322 | AAACAUGAAUUGCUGCUGUAUC | GAUACAGCAGCAAUUCAUGUUU |
| hsa-miR-323 | GCACAUUACACGGUCGACCUCU | AGAGGUCGACCGUGUAAUGUGC |
| hsa-miR-324-3p | CCACUGCCCCAGGUGCUGCUGG | CCAGCAGCACCUGGGGCAGUGG |
| hsa-miR-324-5p | CGCAUCCCCUAGGGCAUUGGUG | CACCAAUGCCCUAGGGGAUGCG |
| hsa-miR-326 | CCUCUGGGCCCUUCCUCCAGCC | GGCUGGAGGAAGGGCCCAGAGG |
| hsa-miR-328 | CUGGCCCUCUCUGCCCUUCCGU | ACGGAAGGGCAGAGAGGGCCAG |
| hsa-miR-329 | AACACACCCAGCUAACCUUUUU | AAAAAGGUUAGCUGGGUGUGUU |
| hsa-miR-34a | UGGCAGUGUCUUAGCUGGUUGU | ACAACCAGCUAAGACACUGCCA |
| hsa-miR-34b | AGGCAGUGUCAUUAGCUGAUUG | CAAUCAGCUAAUGACACUGCCU |
| hsa-miR-34c | AGGCAGUGUAGUUAGCUGAUUG | CAAUCAGCUAACUACACUGCCU |
| hsa-miR-92 | UAUUGCACUUGUCCCGGCCUGU | ACAGGCCGGGACAAGUGCAAUA |
| hsa-miR-93 | AAAGUGCUGUUCGUGCAGGUAG | CUACCUGCACGAACAGCACUUU |
| hsa-miR-95 | UUCAACGGGUAUUUAUUGAGCA | UGCUCAAUAAAUACCCGUUGAA |
| hsa-miR-96 | UUUGGCACUAGCACAUUUUUGC | GCAAAAAUGUGCUAGUGCCAAA |
| hsa-miR-98 | UGAGGUAGUAAGUUGUAUUGUU | AACAAUACAACUUACUACCUCA |
| mmu-miR-106a | CAAAGUGCUAACAGUGCAGGUA | UACCUGCACUGUUAGCACUUUG |
| mmu-miR-10b | CCCUGUAGAACCGAAUUUGUGU | ACACAAAUUCGGUUCUACAGGG |
| mmu-miR-135b | UAUGGCUUUUCAUUCCUAUGUG | CACAUAGGAAUGAAAAGCCAUA |
| mmu-miR-148b | UCAGUGCAUCACAGAACUUUGU | ACAAAGUUCUGUGAUGCACUGA |
| mmu-miR-151-3p | CUAGACUGAGGCUCCUUGAGGA | UCCUCAAGGAGCCUCAGUCUAG |
| mmu-miR-155 | UUAAUGCUAAUUGUGAUAGGGG | CCCCUAUCACAAUUAGCAUUAA |
| mmu-miR-199b | CCCAGUGUUUAGACUACCUGUU | AACAGGUAGUCUAAACACUGGG |
| mmu-miR-200b | UAAUACUGCCUGGUAAUGAUGA | UCAUCAUUACCAGGCAGUAUUA |
| mmu-miR-203 | UGAAAUGUUUAGGACCACUAGA | UCUAGUGGUCCUAAACAUUUCA |
| mmu-miR-211 | UUCCCUUUGUCAUCCUUUGCCU | AGGCAAAGGAUGACAAAGGGAA |
| mmu-miR-217 | UACUGCAUCAGGAACUGACUGG | CCAGUCAGUUCCUGAUGCAGUA |
| mmu-miR-224 | UAAGUCACUAGUGGUUCCGUUU | AAACGGAACCACUAGUGACUUA |
| mmu-miR-28-3p | CACUAGAUUGUGAGCUGCUGGA | UCCAGCAGCUCACAAUCUAGUG |
| mmu-miR-290 | CUCAAACUAUGGGGGCACUUUU | AAAAGUGCCCCCAUAGUUUGAG |
| mmu-miR-291-3p | AAAGUGCUUCCACUUUGUGUGC | GCACACAAAGUGGAAGCACUUU |
| mmu-miR-291-5p | CAUCAAAGUGGAGGCCCUCUCU | AGAGAGGGCCUCCACUUUGAUG |
| mmu-miR-292-3p | AAGUGCCGCCAGGUUUUGAGUG | CACUCAAAACCUGGCGGCACUU |
| mmu-miR-292-5p | ACUCAAACUGGGGGCUCUUUUG | CAAAAGAGCCCCCAGUUUGAGU |
| mmu-miR-293 | AGUGCCGCAGAGUUUGUAGUGU | ACACUACAAACUCUGCGGCACU |
| mmu-miR-294 | AAAGUGCUUCCCUUUUGUGUGU | ACACACAAAAGGGAAGCACUUU |
| mmu-miR-295 | AAAGUGCUACUACUUUUGAGUC | GACUCAAAAGUAGUAGCACUUU |
| mmu-miR-297 | AUGUAUGUGUGCAUGUGCAUGU | ACAUGCACAUGCACACAUACAU |
| mmu-miR-298 | GGCAGAGGAGGGCUGUUCUUCC | GGAAGAACAGCCCUCCUCUGCC |
| mmu-miR-300 | UAUGCAAGGGCAAGCUCUCUUC | GAAGAGAGCUUGCCCUUGCAUA |
| mmu-miR-31 | AGGCAAGAUGCUGGCAUAGCUG | CAGCUAUGCCAGCAUCUUGCCU |
| mmu-miR-322 | AAACAUGAAGCGCUGCAACACC | GGUGUUGCAGCGCUUCAUGUUU |
| mmu-miR-325 | CCUAGUAGGUGCUCAGUAAGUG | CACUUACUGAGCACCUACUAGG |
| mmu-miR-326 | CCUCUGGGCCCUUCCUCCAGUC | GACUGGAGGAAGGGCCCAGAGG |
| mmu-miR-330 | GCAAAGCACAGGGCCUGCAGAG | CUCUGCAGGCCCUGUGCUUUGC |
| mmu-miR-331 | GCCCCUGGGCCUAUCCUAGAAC | GUUCUAGGAUAGGCCCAGGGGC |
| mmu-miR-337 | UUCAGCUCCUAUAUGAUGCCUU | AAGGCAUCAUAUAGGAGCUGAA |
| mmu-miR-338 | UCCAGCAUCAGUGAUUUUGUUG | CAACAAAAUCACUGAUGCUGGA |
| mmu-miR-339 | UCCCUGUCCUCCAGGAGCUCAC | GUGAGCUCCUGGAGGACAGGGA |
| mmu-miR-340 | UCCGUCUCAGUUACUUUAUAGC | GCUAUAAAGUAACUGAGACGGA |
| mmu-miR-341 | UCGAUCGGUCGGUCGGUCAGUC | GACUGACCGACCGACCGAUCGA |
| mmu-miR-342 | UCUCACACAGAAAUCGCACCCG | CGGGUGCGAUUUCUGUGUGAGA |
| mmu-miR-344 | UGAUCUAGCCAAAGCCUGACUG | CAGUCAGGCUUUGGCUAGAUCA |
| mmu-miR-345 | UGCUGACCCCUAGUCCAGUGCU | AGCACUGGACUAGGGGUCAGCA |
| mmu-miR-346 | UGUCUGCCCGAGUGCCUGCCUC | GAGGCAGGCACUCGGGCAGACA |
| mmu-miR-34b | UAGGCAGUGUAAUUAGCUGAUU | AAUCAGCUAAUUACACUGCCUA |
| mmu-miR-350 | UUCACAAAGCCCAUACACUUUC | GAAAGUGUAUGGGCUUUGUGAA |
| mmu-miR-351 | UCCCUGAGGAGCCCUUUGAGCC | GGCUCAAAGGGCUCCUCAGGGA |
| mmu-miR-7b | UGGAAGACUUGUGAUUUUGUUG | CAACAAAAUCACAAGUCUUCCA |
| mmu-miR-92 | UAUUGCACUUGUCCCGGCCUGA | UCAGGCCGGGACAAGUGCAAUA |
| mmu-miR-93 | CAAAGUGCUGUUCGUGCAGGUA | UACCUGCACGAACAGCACUUUG |
| rno-miR-327 | CCUUGAGGGGCAUGAGGGUAGU | ACUACCCUCAUGCCCCUCAAGG |
| rno-miR-333 | GUGGUGUGCUAGUUACUUUUGG | CCAAAAGUAACUAGCACACCAC |
| rno-miR-335 | UCAAGAGCAAUAACGAAAAAUG | CAUUUUUCGUUAUUGCUCUUGA |
| rno-miR-336 | UCACCCUUCCAUAUCUAGUCUC | GAGACUAGAUAUGGAAGGGUGA |
| rno-miR-343 | UCUCCCUCCGUGUGCCCAGUAU | AUACUGGGCACACGGAGGGAGA |
| rno-miR-347 | UGUCCCUCUGGGUCGCCCAGCU | AGCUGGGCGACCCAGAGGGACA |
| rno-miR-349 | CAGCCCUGCUGUCUUAACCUCU | AGAGGUUAAGACAGCAGGGCUG |
| rno-miR-352 | AGAGUAGUAGGUUGCAUAGUAC | GUACUAUGCAACCUACUACUCU |

TABLE 2

Novel Human microRNA and anti-microRNA sequences.

| | |
|---|---|
| microRNA sequence | Anti-microRNA molecule |

| | | |
|---|---|---|
| microRNA name | (5′ to 3′) | sequence (5′ to 3′) |

| | | |
|---|---|---|
| hsa-miR-361 | UUAUCAGAAUCUCCAGGGGUAC | GUACCCCUGGAGAUUCUGAUAA |
| hsa-miR-362 | AAUCCUUGGAACCUAGGUGUGA | UCACACCUAGGUUCCAAGGAUU |
| hsa-miR-363 | AUUGCACGGUAUCCAUCUGUAA | UUACAGAUGGAUACCGUGCAAU |
| hsa-miR-364 | CGGCGGGGACGGCGAUUGGUCC | GGACCAAUCGCCGUCCCCGCCG |
| hsa-miR-365 | UAAUGCCCCUAAAAAUCCUUAU | AUAAGGAUUUUUAGGGGCAUUA |
| hsa-miR-366 | UAACUGGUUGAACAACUGAACC | GGUUCAGUUGUUCAACCAGUUA |

TABLE 3

C. elegans microRNA and anti-microRNA sequences.

| | |
|---|---|
| microRNA sequence | Anti-microRNA molecule |

| | | |
|---|---|---|
| microRNA name | (5′ to 3′) | sequence (5′ to 3′) |

| | | |
|---|---|---|
| Cel-let-7 | UGAGGUAGUAGGUUGUAUAGUU | AACUAUACAACCUACUACCUCA |
| Cel-lin-4 | UCCCUGAGACCUCAAGUGUGAG | CUCACACUUGAGGUCUCAGGGA |
| Cel-miR-1 | UGGAAUGUAAAGAAGUAUGUAG | CUACAUACUUCUUUACAUUCCA |
| Cel-miR-2 | UAUCACAGCCAGCUUUGAUGUG | CACAUCAAAGCUGGCUGUGAUA |
| Cel-miR-34 | AGGCAGUGUGGUUAGCUGGUUG | CAACCAGCUAACCACACUGCCU |
| Cel-miR-35 | UCACCGGGUGGAAACUAGCAGU | ACUGCUAGUUUCCACCCGGUGA |
| Cel-miR-36 | UCACCGGGUGAAAAUUCGCAUG | CAUGCGAAUUUUCACCCGGUGA |
| Cel-miR-37 | UCACCGGGUGAACACUUGCAGU | ACUGCAAGUGUUCACCCGGUGA |
| Cel-miR-38 | UCACCGGGAGAAAAACUGGAGU | ACUCCAGUUUUUCUCCCGGUGA |
| Cel-miR-39 | UCACCGGGUGUAAAUCAGCUUG | CAAGCUGAUUUACACCCGGUGA |
| Cel-miR-40 | UCACCGGGUGUACAUCAGCUAA | UUAGCUGAUGUACACCCGGUGA |
| Cel-miR-41 | UCACCGGGUGAAAAAUCACCUA | UAGGUGAUUUUUCACCCGGUGA |
| Cel-miR-42 | CACCGGGUUAACAUCUACAGAG | CUCUGUAGAUGUUAACCCGGUG |
| Cel-miR-43 | UAUCACAGUUUACUUGCUGUCG | CGACAGCAAGUAAACUGUGAUA |
| Cel-miR-44 | UGACUAGAGACACAUUCAGCUU | AAGCUGAAUGUGUCUCUAGUCA |
| Cel-miR-45 | UGACUAGAGACACAUUCAGCUU | AAGCUGAAUGUGUCUCUAGUCA |
| Cel-miR-46 | UGUCAUGGAGUCGCUCUCUUCA | UGAAGAGAGCGACUCCAUGACA |
| Cel-miR-47 | UGUCAUGGAGGCGCUCUCUUCA | UGAAGAGAGCGCCUCCAUGACA |
| Cel-miR-48 | UGAGGUAGGCUCAGUAGAUGCG | CGCAUCUACUGAGCCUACCUCA |
| Cel-miR-49 | AAGCACCACGAGAAGCUGCAGA | UCUGCAGCUUCUCGUGGUGCUU |
| Cel-miR-50 | UGAUAUGUCUGGUAUUCUUGGG | CCCAAGAAUACCAGACAUAUCA |
| Cel-miR-51 | UACCCGUAGCUCCUAUCCAUGU | ACAUGGAUAGGAGCUACGGGUA |
| Cel-miR-52 | CACCCGUACAUAUGUUUCCGUG | CACGGAAACAUAUGUACGGGUG |
| Cel-miR-53 | CACCCGUACAUUUGUUUCCGUG | CACGGAAACAAAUGUACGGGUG |
| Cel-miR-54 | UACCCGUAAUCUUCAUAAUCCG | CGGAUUAUGAAGAUUACGGGUA |
| Cel-miR-55 | UACCCGUAUAAGUUUCUGCUGA | UCAGCAGAAACUUAUACGGGUA |
| Cel-miR-56 | UACCCGUAAUGUUUCCGCUGAG | CUCAGCGGAAACAUUACGGGUA |
| Cel-miR-57 | UACCCUGUAGAUCGAGCUGUGU | ACACAGCUCGAUCUACAGGGUA |
| Cel-miR-58 | UGAGAUCGUUCAGUACGGCAAU | AUUGCCGUACUGAACGAUCUCA |
| Cel-miR-59 | UCGAAUCGUUUAUCAGGAUGAU | AUCAUCCUGAUAAACGAUUCGA |
| Cel-miR-60 | UAUUAUGCACAUUUUCUAGUUC | GAACUAGAAAAUGUGCAUAAUA |
| Cel-miR-61 | UGACUAGAACCGUUACUCAUCU | AGAUGAGUAACGGUUCUAGUCA |
| Cel-miR-62 | UGAUAUGUAAUCUAGCUUACAG | CUGUAAGCUAGAUUACAUAUCA |
| Cel-miR-63 | AUGACACUGAAGCGAGUUGGAA | UUCCAACUCGCUUCAGUGUCAU |
| Cel-miR-64 | UAUGACACUGAAGCGUUACCGA | UCGGUAACGCUUCAGUGUCAUA |
| Cel-miR-65 | UAUGACACUGAAGCGUAACCGA | UCGGUUACGCUUCAGUGUCAUA |
| Cel-miR-66 | CAUGACACUGAUUAGGGAUGUG | CACAUCCCUAAUCAGUGUCAUG |
| Cel-miR-67 | UCACAACCUCCUAGAAAGAGUA | UACUCUUUCUAGGAGGUUGUGA |
| Cel-miR-68 | UCGAAGACUCAAAAGUGUAGAC | GUCUACACUUUUGAGUCUUCGA |
| Cel-miR-69 | UCGAAAAUUAAAAAGUGUAGAA | UUCUACACUUUUUAAUUUUCGA |
| Cel-miR-70 | UAAUACGUCGUUGGUGUUUCCA | UGGAAACACCAACGACGUAUUA |
| Cel-miR-71 | UGAAAGACAUGGGUAGUGAACG | CGUUCACUACCCAUGUCUUUCA |
| Cel-miR-72 | AGGCAAGAUGUUGGCAUAGCUG | CAGCUAUGCCAACAUCUUGCCU |
| Cel-miR-73 | UGGCAAGAUGUAGGCAGUUCAG | CUGAACUGCCUACAUCUUGCCA |
| Cel-miR-74 | UGGCAAGAAAUGGCAGUCUACA | UGUAGACUGCCAUUUCUUGCCA |
| Cel-miR-75 | UUAAAGCUACCAACCGGCUUCA | UGAAGCCGGUUGGUAGCUUUAA |
| Cel-miR-76 | UUCGUUGUUGAUGAAGCCUUGA | UCAAGGCUUCAUCAACAACGAA |
| Cel-miR-77 | UUCAUCAGGCCAUAGCUGUCCA | UGGACAGCUAUGGCCUGAUGAA |
| Cel-miR-78 | UGGAGGCCUGGUUGUUUGUGCU | AGCACAAACAACCAGGCCUCCA |
| Cel-miR-79 | AUAAAGCUAGGUUACCAAAGCU | AGCUUUGGUAACCUAGCUUUAU |
| Cel-miR-227 | AGCUUUCGACAUGAUUCUGAAC | GUUCAGAAUCAUGUCGAAAGCU |
| Cel-miR-80 | UGAGAUCAUUAGUUGAAAGCCG | CGGCUUUCAACUAAUGAUCUCA |
| Cel-miR-81 | UGAGAUCAUCGUGAAAGCUAGU | ACUAGCUUUCACGAUGAUCUCA |
| Cel-miR-82 | UGAGAUCAUCGUGAAAGCCAGU | ACUGGCUUUCACGAUGAUCUCA |
| Cel-miR-83 | UAGCACCAUAUAAAUUCAGUAA | UUACUGAAUUUAUAUGGUGCUA |
| Cel-miR-84 | UGAGGUAGUAUGUAAUAUUGUA | UACAAUAUUACAUACUACCUCA |
| Cel-miR-85 | UACAAAGUAUUUGAAAAGUCGU | ACGACUUUUCAAAUACUUUGUA |
| Cel-miR-86 | UAAGUGAAUGCUUUGCCACAGU | ACUGUGGCAAAGCAUUCACUUA |
| Cel-miR-87 | GUGAGCAAAGUUUCAGGUGUGC | GCACACCUGAAACUUUGCUCAC |
| Cel-miR-90 | UGAUAUGUUGUUUGAAUGCCCC | GGGGCAUUCAAACAACAUAUCA |
| Cel-miR-124 | UAAGGCACGCGGUGAAUGCCAC | GUGGCAUUCACCGCGUGCCUUA |
| Cel-miR-228 | AAUGGCACUGCAUGAAUUCACG | CGUGAAUUCAUGCAGUGCCAUU |
| Cel-miR-229 | AAUGACACUGGUUAUCUUUUCC | GGAAAAGAUAACCAGUGUCAUU |
| Cel-miR-230 | GUAUUAGUUGUGCGACCAGGAG | CUCCUGGUCGCACAACUAAUAC |
| Cel-miR-231 | UAAGCUCGUGAUCAACAGGCAG | CUGCCUGUUGAUCACGAGCUUA |
| Cel-miR-232 | UAAAUGCAUCUUAACUGCGGUG | CACCGCAGUUAAGAUGCAUUUA |
| Cel-miR-233 | UUGAGCAAUGCGCAUGUGCGGG | CCCGCACAUGCGCAUUGCUCAA |
| Cel-miR-234 | UUAUUGCUCGAGAAUACCCUUU | AAAGGGUAUUCUCGAGCAAUAA |
| Cel-miR-235 | UAUUGCACUCUCCCCGGCCUGA | UCAGGCCGGGGAGAGUGCAAUA |
| Cel-miR-236 | UAAUACUGUCAGGUAAUGACGC | GCGUCAUUACCUGACAGUAUUA |
| Cel-miR-237 | UCCCUGAGAAUUCUCGAACAGC | GCUGUUCGAGAAUUCUCAGGGA |
| Cel-miR-238 | UUUGUACUCCGAUGCCAUUCAG | CUGAAUGGCAUCGGAGUACAAA |
| Cel-miR-239a | UUUGUACUACACAUAGGUACUG | CAGUACCUAUGUGUAGUACAAA |
| Cel-miR-239b | UUUGUACUACACAAAAGUACUG | CAGUACUUUUGUGUAGUACAAA |
| Cel-miR-240 | UACUGGCCCCCAAAUCUUCGCU | AGCGAAGAUUUGGGGGCCAGUA |
| Cel-miR-241 | UGAGGUAGGUGCGAGAAAUGAC | GUCAUUUCUCGCACCUACCUCA |
| Cel-miR-242 | UUGCGUAGGCCUUUGCUUCGAG | CUCGAAGCAAAGGCCUACGCAA |
| Cel-miR-243 | CGGUACGAUCGCGGCGGGAUAU | AUAUCCCGCCGCGAUCGUACCG |
| Cel-miR-244 | UCUUUGGUUGUACAAAGUGGUA | UACCACUUUGUACAACCAAAGA |
| Cel-miR-245 | AUUGGUCCCCUCCAAGUAGCUC | GAGCUACUUGGAGGGGACCAAU |
| Cel-miR-246 | UUACAUGUUUCGGGUAGGAGCU | AGCUCCUACCCGAAACAUGUAA |
| Cel-miR-247 | UGACUAGAGCCUAUUCUCUUCU | AGAAGAGAAUAGGCUCUAGUCA |
| Cel-miR-248 | UACACGUGCACGGAUAACGCUC | GAGCGUUAUCCGUGCACGUGUA |
| Cel-miR-249 | UCACAGGACUUUUGAGCGUUGC | GCAACGCUCAAAAGUCCUGUGA |
| Cel-miR-250 | UCACAGUCAACUGUUGGCAUGG | CCAUGCCAACAGUUGACUGUGA |
| Cel-miR-251 | UUAAGUAGUGGUGCCGCUCUUA | UAAGAGCGGCACCACUACUUAA |
| Cel-miR-252 | UAAGUAGUAGUGCCGCAGGUAA | UUACCUGCGGCACUACUACUUA |
| Cel-miR-253 | CACACCUCACUAACACUGACCA | UGGUCAGUGUUAGUGAGGUGUG |
| Cel-miR-254 | UGCAAAUCUUUCGCGACUGUAG | CUACAGUCGCGAAAGAUUUGCA |
| Cel-miR-256 | UGGAAUGCAUAGAAGACUGUAC | GUACAGUCUUCUAUGCAUUCCA |
| Cel-miR-257 | GAGUAUCAGGAGUACCCAGUGA | UCACUGGGUACUCCUGAUACUC |
| Cel-miR-258 | GGUUUUGAGAGGAAUCCUUUUA | UAAAAGGAUUCCUCUCAAAACC |
| Cel-miR-259 | AGUAAAUCUCAUCCUAAUCUGG | CCAGAUUAGGAUGAGAUUUACU |
| Cel-miR-260 | GUGAUGUCGAACUCUUGUAGGA | UCCUACAAGAGUUCGACAUCAC |
| Cel-miR-261 | UAGCUUUUUAGUUUUCACGGUG | CACCGUGAAAACUAAAAAGCUA |
| Cel-miR-262 | GUUUCUCGAUGUUUUCUGAUAC | GUAUCAGAAAACAUCGAGAAAC |
| Cel-miR-264 | GGCGGGUGGUUGUUGUUAUGGG | CCCAUAACAACAACCACCCGCC |
| Cel-miR-265 | UGAGGGAGGAAGGGUGGUAUUU | AAAUACCACCCUUCCUCCCUCA |
| Cel-miR-266 | AGGCAAGACUUUGGCAAAGCUU | AAGCUUUGCCAAAGUCUUGCCU |
| Cel-miR-267 | CCCGUGAAGUGUCUGCUGCAAU | AUUGCAGCAGACACUUCACGGG |
| Cel-miR-268 | GGCAAGAAUUAGAAGCAGUUUG | CAAACUGCUUCUAAUUCUUGCC |
| Cel-miR-269 | GGCAAGACUCUGGCAAAACUUG | CAAGUUUUGCCAGAGUCUUGCC |
| Cel-miR-270 | GGCAUGAUGUAGCAGUGGAGAU | AUCUCCACUGCUACAUCAUGCC |
| Cel-miR-271 | UCGCCGGGUGGGAAAGCAUUCG | CGAAUGCUUUCCCACCCGGCGA |
| Cel-miR-272 | UGUAGGCAUGGGUGUUUGGAAG | CUUCCAAACACCCAUGCCUACA |
| Cel-miR-273 | UGCCCGUACUGUGUCGGCUGCU | AGCAGCCGACACAGUACGGGCA |

TABLE 4

Drosophila microRNA and anti-microRNA sequences.

| | |
|---|---|
| microRNA sequence | Anti-microRNA molecule |

| | | |
|---|---|---|
| microRNA name | (5′ to 3′) | sequence (5′ to 3′) |

| | | |
|---|---|---|
| Dme-miR-263a | GUUAAUGGCACUGGAAGAAUUC | GAAUUCUUCCAGUGCCAUUAAC |
| Dme-miR-184 | UGGACGGAGAACUGAUAAGGGC | GCCCUUAUCAGUUCUCCGUCCA |
| Dme-miR-274 | UUUUGUGACCGACACUAACGGG | CCCGUUAGUGUCGGUCACAAAA |
| Dme-miR-275 | UCAGGUACCUGAAGUAGCGCGC | GCGCGCUACUUCAGGUACCUGA |
| Dme-miR-92a | CAUUGCACUUGUCCCGGCCUAU | AUAGGCCGGGACAAGUGCAAUG |
| Dme-miR-219 | UGAUUGUCCAAACGCAAUUCUU | AAGAAUUGCGUUUGGACAAUCA |
| Dme-miR-276a | UAGGAACUUCAUACCGUGCUCU | AGAGCACGGUAUGAAGUUCCUA |
| Dme-miR-277 | UAAAUGCACUAUCUGGUACGAC | GUCGUACCAGAUAGUGCAUUUA |
| Dme-miR-278 | UCGGUGGGACUUUCGUCCGUUU | AAACGGACGAAAGUCCCACCGA |
| Dme-miR-133 | UUGGUCCCCUUCAACCAGCUGU | ACAGCUGGUUGAAGGGGACCAA |
| Dme-miR-279 | UGACUAGAUCCACACUCAUUAA | UUAAUGAGUGUGGAUCUAGUCA |
| Dme-miR-33 | AGGUGCAUUGUAGUCGCAUUGU | ACAAUGCGACUACAAUGCACCU |
| Dme-miR-280 | UGUAUUUACGUUGCAUAUGAAA | UUUCAUAUGCAACGUAAAUACA |
| Dme-miR-281 | UGUCAUGGAAUUGCUCUCUUUG | CAAAGAGAGCAAUUCCAUGACA |
| Dme-miR-282 | AAUCUAGCCUCUACUAGGCUUU | AAAGCCUAGUAGAGGCUAGAUU |
| Dme-miR-283 | UAAAUAUCAGCUGGUAAUUCUG | CAGAAUUACCAGCUGAUAUUUA |
| Dme-miR-284 | UGAAGUCAGCAACUUGAUUCCA | UGGAAUCAAGUUGCUGACUUCA |
| Dme-miR-34 | UGGCAGUGUGGUUAGCUGGUUG | CAACCAGCUAACCACACUGCCA |
| Dme-miR-124 | UAAGGCACGCGGUGAAUGCCAA | UUGGCAUUCACCGCGUGCCUUA |
| Dme-miR-79 | UAAAGCUAGAUUACCAAAGCAU | AUGCUUUGGUAAUCUAGCUUUA |
| Dme-miR-276b | UAGGAACUUAAUACCGUGCUCU | AGAGCACGGUAUUAAGUUCCUA |
| Dme-miR-210 | UUGUGCGUGUGACAGCGGCUAU | AUAGCCGCUGUCACACGCACAA |
| Dme-miR-285 | UAGCACCAUUCGAAAUCAGUGC | GCACUGAUUUCGAAUGGUGCUA |
| Dme-miR-100 | AACCCGUAAAUCCGAACUUGUG | CACAAGUUCGGAUUUACGGGUU |
| Dme-miR-92b | AAUUGCACUAGUCCCGGCCUGC | GCAGGCCGGGACUAGUGCAAUU |
| Dme-miR-286 | UGACUAGACCGAACACUCGUGC | GCACGAGUGUUCGGUCUAGUCA |
| Dme-miR-287 | UGUGUUGAAAAUCGUUUGCACG | CGUGCAAACGAUUUUCAACACA |
| Dme-miR-87 | UUGAGCAAAAUUUCAGGUGUGU | ACACACCUGAAAUUUUGCUCAA |
| Dme-miR-263b | CUUGGCACUGGGAGAAUUCACA | UGUGAAUUCUCCCAGUGCCAAG |
| Dme-miR-288 | UUUCAUGUCGAUUUCAUUUCAU | AUGAAAUGAAAUCGACAUGAAA |
| Dme-miR-289 | UAAAUAUUUAAGUGGAGCCUGC | GCAGGCUCCACUUAAAUAUUUA |
| Dme-bantam | UGAGAUCAUUUUGAAAGCUGAU | AUCAGCUUUCAAAAUGAUCUCA |
| Dme-miR-303 | UUUAGGUUUCACAGGAAACUGG | CCAGUUUCCUGUGAAACCUAAA |
| Dme-miR-31b | UGGCAAGAUGUCGGAAUAGCUG | CAGCUAUUCCGACAUCUUGCCA |
| Dme-miR-304 | UAAUCUCAAUUUGUAAAUGUGA | UCACAUUUACAAAUUGAGAUUA |
| Dme-miR-305 | AUUGUACUUCAUCAGGUGCUCU | AGAGCACCUGAUGAAGUACAAU |
| Dme-miR-9c | UCUUUGGUAUUCUAGCUGUAGA | UCUACAGCUAGAAUACCAAAGA |
| Dme-miR-306 | UCAGGUACUUAGUGACUCUCAA | UUGAGAGUCACUAAGUACCUGA |
| Dme-miR-9b | UCUUUGGUGAUUUUAGCUGUAU | AUACAGCUAAAAUCACCAAAGA |
| Dme-miR-125 | UCCCUGAGACCCUAACUUGUGA | UCACAAGUUAGGGUCUCAGGGA |
| Dme-miR-307 | UCACAACCUCCUUGAGUGAGCG | CGCUCACUCAAGGAGGUUGUGA |
| Dme-miR-308 | AAUCACAGGAUUAUACUGUGAG | CUCACAGUAUAAUCCUGUGAUU |
| dme-miR-31a | UGGCAAGAUGUCGGCAUAGCUG | CAGCUAUGCCGACAUCUUGCCA |
| dme-miR-309 | GCACUGGGUAAAGUUUGUCCUA | UAGGACAAACUUUACCCAGUGC |
| dme-miR-310 | UAUUGCACACUUCCCGGCCUUU | AAAGGCCGGGAAGUGUGCAAUA |
| dme-miR-311 | UAUUGCACAUUCACCGGCCUGA | UCAGGCCGGUGAAUGUGCAAUA |
| dme-miR-312 | UAUUGCACUUGAGACGGCCUGA | UCAGGCCGUCUCAAGUGCAAUA |
| dme-miR-313 | UAUUGCACUUUUCACAGCCCGA | UCGGGCUGUGAAAAGUGCAAUA |
| dme-miR-314 | UAUUCGAGCCAAUAAGUUCGG | CCGAACUUAUUGGCUCGAAUA |
| dme-miR-315 | UUUUGAUUGUUGCUCAGAAAGC | GCUUUCUGAGCAACAAUCAAAA |
| dme-miR-316 | UGUCUUUUUCCGCUUACUGGCG | CGCCAGUAAGCGGAAAAAGACA |
| dme-miR-317 | UGAACACAGCUGGUGGUAUCCA | UGGAUACCACCAGCUGUGUUCA |
| dme-miR-318 | UCACUGGGCUUUGUUUAUCUCA | UGAGAUAAACAAAGCCCAGUGA |
| dme-miR-2c | UAUCACAGCCAGCUUUGAUGGG | CCCAUCAAAGCUGGCUGUGAUA |
| Dme-miR-iab45p | ACGUAUACUGAAUGUAUCCUGA | UCAGGAUACAUUCAGUAUACGU |
| Dme-miR-iab43p | CGGUAUACCUUCAGUAUACGUA | UACGUAUACUGAAGGUAUACCG |

### EXAMPLES

### Example 1

### Materials and Methods

Oligonucleotide synthesis

MiR-21 were synthesized using 5′-silyl, 2′-ACE phosphoramidites (Dharmacon, Lafayette, Colo., USA) on 0.2 μmol synthesis columns using a modified ABI 394 synthesizer (Foster City, Calif., USA) (Scaringe, Methods Enzymol. 317, 3-18 (2001) and Scaringe, Methods 23, 206-217 (2001)). The phosphate methyl group was removed by flushing the column with 2 ml of 0.2 M 2-carbamoyl-2-cyanoethylene-1,1-dithiolate trihydrate in DMF/water (98:2 v/v) for 30 min at room temperature. The reagent was removed and the column rinsed with 10 ml water followed by 10 ml acetonitrile. The oligonucleotide was cleaved and eluted from the solid support by flushing with 1.6 ml of 40% aqueous methylamine over 2 min, collected in a screwcap vial and incubated for 10 min at 55° C. Subsequently, the base-treated oligonucleotide was dried down in an Eppendorf concentrator to remove methylamine and water. The residue was dissolved in sterile 2′-deprotection buffer (400 μl of 100 mM acetate-TEMED, pH 3.8, for a 0.2 μmol scale synthesis) and incubated for 30 minutes at 60° C. to remove the 2′ ACE group. The oligoribonucleotide was precipitated from the acetate-TEMED solution by adding 24 μl 5 M NaCl and 1.2 ml of absolute ethanol.

2′-O-Methyl oligoribonucleotides were synthesized using 5′-DMT, 2′-O-methyl phosphoramidites (Proligo, Hamburg, Germany) on 1 μmol synthesis columns loaded with 3′-aminomodifier (TFA) C7 Icaa control pore glass support (Chemgenes, Mass., USA). The aminolinker was added in order to also use the oligonucleotides for conjugation to amino group reactive reagents, such as biotin succinimidyl esters. The synthesis products were deprotected for 16 h at 55° C. in 30% aqueous ammonia and then precipitated by the addition of 12 ml absolute 1-butanol. The full-length product was then gel-purified using a denaturing 20% polyacrylamide gel. 2′-Deoxyoligonucleotides were prepared using 0.2 μmol scale synthesis and standard DNA synthesis reagents (Proligo, Hamburg, Germany).

The sequences of the 2′-O-methyl oligoribonucleotides were 5′-GUCAACAUCAGUCUGAUAAGCUAL (L, 3′ aminolinker) for 2′-OMe miR-21, and 5′-AAGGCAAGCUGACCCUGAAGUL for EGFP 2′-OMe antisense, 5′-UGAAGUCCCAGUCGAACGGAAL for EGFP 2′-OMe reverse; the sequence of chimeric 2′-OMe/DNA oligonucleotides was 5′-GTCAACATCAGTCTGATAAGCTAGCGL for 2′-deoxy miR-21 (underlined, 2′-OMe residues), and 5′-AAGGCAAGCTGACCCTGAAGTGCGL for EGFP 2′-deoxy antisense.

The miR-2 1 cleavage substrate was prepared by PCR-based extension of the partially complementary synthetic DNA oligonucleotides 5′-GAACAATTGCTTTTACAGATGCACATATCGAGGTGAACATCACGTACGTCAACATCA GTCTGATAAGCTATCGGTTGGCAGAAGCTAT and 5′-GGCATAAAGAATTGAAGAGAGTTTTCACTGCATACGACGATTCTGTGATTTGTATTC AGCCCATATCGTTTCATAGCTTCTGCCAACCGA. The extended dsDNA was then used as template for a new PCR with primers 5′-TAATACGACTCACTATAGAACAATTGCTTTTACAG and 5′-ATTTAGGTGACACTATAGGCATAAAGAATTGAAGA to introduce the T7 and SP6 promoter sequences for in vitro transcription. The PCR product was ligated into pCR2.1-TOPO (Invitrogen). Plasmids isolated from sequence-verified clones were used as templates for PCR to produce sufficient template for run-off in vitro transcription reactions using phage RNA polymerases (Elbashir et al., EMBO 20, 6877-6888 (2001)). ₃₂P-Cap-labelling was performed as reported (Martinez et al., Cell 110, 563-574 (2002)).

Plasmids

Plasmids pEGFP-S-21 and pEGFP-A-21 were generated by T4 DNA ligation of preannealed oligodeoxynucleotides 5′-GGCCTCAACATCAGTCTGATAAGCTAGGTACCT and 5′-GGCCAGGTACCTAGCTTATCAGACTGATGTTGA into NotI digested pEGFP-N-1 (Clontech). The plasmid pHcRed-C1 was from Clontech.

HeLa Extracts and miR-21 Quantification

HeLa cell extracts were prepared as described (Dignam et al., Nucleic Acid Res. 11 1475-1489 (1983)). 5×10₉ cells from HeLa suspension cultures were collected by centrifugation and washed with PBS (pH7.4). The cell pellet (approx. 15 ml) was re-suspended in two times of its volume with 10 mM KCl/1.5 mM MgCl₂/0.5 mM dithiothreitol/10 mM HEPES-KOH (pH 7.9) and homogenized by douncing. The nuclei were then removed by centrifugation of the cell lysate at 1000 g for 10 min. The supernatant was spun in an ultracentrifuge for 1 h at 10,5000 g to obtain the cytoplasmic S100 extract. The concentration of KCl of the S100 extract was subsequently raised to 100 mM by the addition of 1 M KCl. The extract was then supplemented with 10% glycerol and frozen in liquid nitrogen.

280 μg of total RNA was isolated from 1 ml of S100 extract using the acidic guanidinium thiocyanate-phenol-chloroform extraction method (Chomczynski et al., Anal. Biochem. 162, 156-159 (1987)). A calibration curve for miR-21 Northern signals was produced by loading increasing amounts (10 to 30000 pg) of synthetically made miR-21 (Lim et al. et al., Genes & Devel. 17, 991-1008 (2003)). Northern blot analysis was performed as described using 30 μg of total RNA per well (Lagos-Quintana et al., Science 294, 853-858 (2001)).

In vitro miRNA cleavage and inhibition assay

2′-O-Methyl oligoribonucleotides or 2′-deoxyoligonucleotides were pre-incubated with HeLa S100 at 30° C. for 20 min prior to the addition of the cap-labeled miR-21 target RNA. The concentration of the reaction components were 5 nM target RNA, 1 mM ATP, 0.2 mM GTP, 10 U/ml RNasin (Promega) and 50% HeLa S100 extract in a final reaction volume of 25 μl. The reaction time was 1.5 h at 30° C. The reaction was stopped by addition of 200 μl of 300 mM NaCl/25 mM EDTA/20% w/v SDS/200 mM Tris HCl (pH7.5). Subsequently, proteinase K was added to a final concentration of 0.6 mg/ml and the sample was incubated for 15 min at 65° C. After phenol/chloroform extraction, the RNA was ethanol-precipitated and separated on a 6% denaturing polyacrylamide gel. Radioactivity was detected by phosphorimaging.

Cell Culture and Transfection

HeLa S3 and HeLa S3/GFP were grown in 5% CO2 at 37° C. in Dulbecco's modified Eagle's medium (DMEM) supplemented with 10% fetal bovine serum (FBS), 100 unit/ml penicillin, and 100 μg/ml streptomycin. One day before transfection, 105 cells were plated in 500 μl DMEM containing 10% FBS per well of a 24-well plate. Plasmid and plasmid/oligonucleotide transfection was carried out with Lipofectamine2000 (Invitrogen). 0.2 μg pEGFP or its derivatives were cotransfected with 0.3 μg pHcRed with or without 10 pmol of 2′-O-methyl oligoribonucleotide or 10 pmol of 2′-deoxyoligonucleotide per well. Fluorescent cell images were recorded on a Zeiss Axiovert 200 inverted fluorescence microscope (Plan-Apochromat 10×/0.45) equipped with Chroma Technology Corp. filter sets 41001 (EGFP) and 41002c (HcRed) and AxioVision 3.1 software.

### Example 2

### MicroRNA-21 Cleavage of Target RNA

In order to assess the ability of modified oligonucleotides to specifically interfere with miRNA function, we used our previously described mammalian biochemical system developed for assaying RISC activity (Martinez et al., Cell 100, 563-574 (2002)). Zamore and colleagues (Hutvágner et al., Science 297, 2056-2050 (2002)) showed that crude cytoplasmic cell lysates and eIF2C2 immunoprecipitates prepared from these lysates contain let-7 RNPs that specifically cleave let-7-complementary target RNAs. We previously reported that in HeLa cells, numerous miRNAs are expressed including several let-7 miRNA variants (Lagos-Quintana et al., Science 294, 853-858 (2001)).

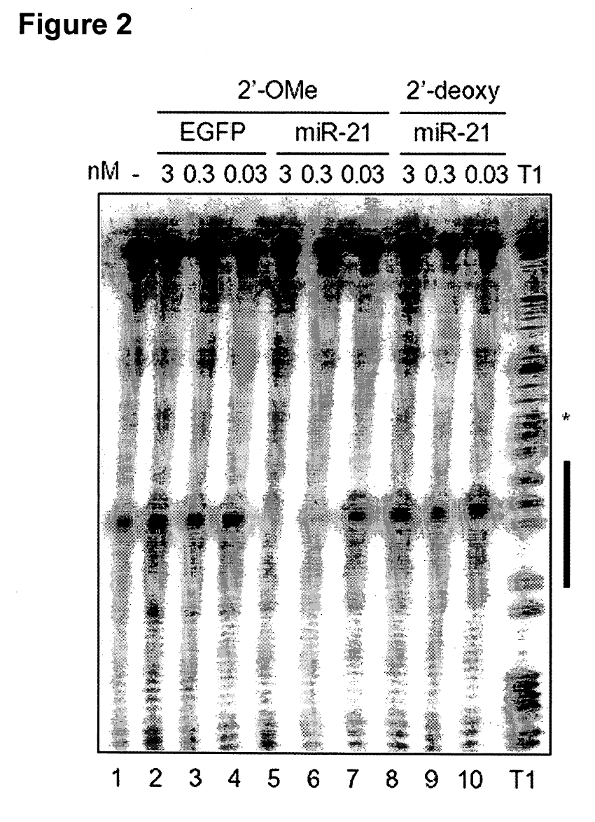

To assess if other HeLa cell miRNAs are also engaged in RISC like miRNPs we examined the cleavage of a 32P-cap-labelled substrate RNA with a complementary site to the highly expressed miR-21 (Lagos-Quintana et al., Science 294, 853-858 (2001); Mourelatos et al., Genes & Dev. 16, 720-728 (2002)). Sequence-specific target RNA degradation was readily observed and appeared to be approximately 2- to 5-fold more effective than cleavage of a similar let-7 target RNA (FIG. 2A, lane 1, and data not shown). We therefore decided to interfere with miR-21 guided target RNA cleavage.

### Example 3

### Anti MicroRNA-21 2′-O-methyl Oligoribonucleotide Inhibited MicroRNA-21-Induced Cleavage of Target RNA

A 24-nucleotide 2′-O-methyl oligoribonucleotide that contained a 3′ C7 aminolinker and was complementary to the longest form of the miR-21 was synthesized. The aminolinker was introduced in order to enable post-synthetic conjugation of non-nucleotidic residues such as biotin.

Increasing concentrations of anti miR-21 2′-O-methyl oligoribonucleotide and a control 2′-O-methyl oligoribonucleotide cognate to an EGFP sequence were added to the S100 extract 20 min prior to the addition of 32P-cap-labelled substrate. We determined the concentration of miR-21 in the S100 extract by quantitative Northern blotting to be 50 pM (Lim et al., Genes & Devel. 17, 991-1008 (2003)).

The control EGFP oligonucleotide did not interfere with miR-21 cleavage even at the highest applied concentration (FIG. 2A, lanes 2-3). In contrast, the activity of miR-21 was completely blocked at a concentration of only 3 nM (FIG. 2A, lane 5), and a concentration of 0.3 nM showed a substantial 60%-70% reduction of cleavage activity (FIG. 2, lane 6). At a concentration of 0.03 nM, the cleavage activity of miR-21 was not affected when compared to the lysate alone (FIG. 2, lane 1, 7).

Antisense 2′-deoxyoligonucleotides (approximately 90% DNA molecules) at concentrations identical to those of 2′-O-methyl oligoribonucleotides, we could not detect blockage of miR-21 induced cleavage (FIG. 2A, lanes 8-10). The 2′-deoxynucleotides used in this study were protected against 3′-exonucleases by the addition of three 2′-O-methyl ribonucleotide residues.

### Example 4

### Anti MicroRNA-21 2′-O-methyl Oligoribonucleotide Inhibited MicroRNA-21-Induced Cleavage of Target RNA In Vitro

In order to monitor the activity of miR-21 in HeLa cells, we constructed reporter plasmids that express EGFP mRNA that contains in its 3′ UTR a 22-nt sequence complementary to miR-21 (pEGFP-S-21) or in sense orientation to miR-21 (p-EGFP-A-21). Endogenous miRNAs have previously been shown to act like siRNAs by cleaving reporter mRNAs carrying sequences perfectly complementary to miRNA. To monitor transfection efficiency and specific interference with the EGFP indicator plasmids, the far-red fluorescent protein encoding plasmid pHcRed-C1 was cotransfected.

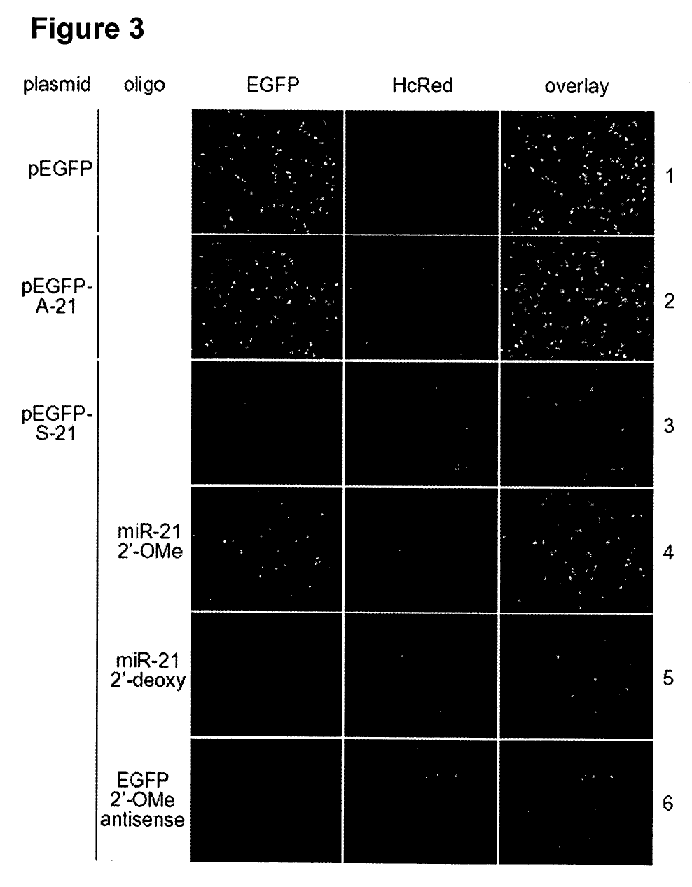

Expression of EGFP was observed in HeLa cells transfected with pEGFP and pEGFP-A-21 (FIG. 3, rows 1 and 2), but not from those transfected with pEGFP-S-21 (FIG. 3, row 3). However, expression of EGFP from pEGFP-S-21 was restored upon cotransfection with anti miR-21 2′-O-methyl oligoribonucleotide (FIG. 3, row 4). Consistent with our above observation, the 2′-deoxy anti miR-21 oligonucleotide showed no effect (FIG. 3, row 5). Similarly, cotransfection of the EGFP 2′-O-methyl oligoribonucleotide in sense orientation with respect to the EGFP mRNA (or antisense to EGFP guide siRNA) had no effect (FIG. 3, row 6).

We have demonstrated that miRNP complexes can be effectively and sequence-specifically inhibited with 2′-O-methyl oligoribonucleotides antisense to the guide strand positioned in the RNA silencing complex.

1. An isolated single stranded anti-microRNA molecule comprising a minimum of ten moieties and a maximum of fifty moieties on a molecular backbone, the molecular backbone comprising backbone units, each moiety comprising a base bonded to a backbone unit, each base forming a Watson-Crick base pair with a complementary base wherein:
at least ten contiguous bases have the same sequence as a sequence of bases in any one of the anti-microRNA molecules shown in Tables 1-4, except that up to thirty percent of the bases pairs may be wobble base pairs, and up to 10% of the contiguous bases may be additions, deletions, mismatches, or combinations thereof;
no more than fifty percent of the contiguous moieties contain deoxyribonuleotide backbone units;
the moiety in the molecule at the position corresponding to position 11 of the microRNA is non-complementary; and
the molecule is capable of inhibiting microRNP activity.

2. A molecule according to claim 1, wherein up to 5% of the contigous moieties are additions, deletions, mismatches, or combinations thereof.

3. A molecule according to claim 1, wherein at least one of the moieties is a deoxyribonucleotide.

4. A molecule according to claim 3, wherein the deoxyribonucleotide is a modified deoxyribonucleotide moiety.

5. A molecule according to claim 4, wherein the modified deoxyribonucleotide is a phosphorothioate deoxyribonucleotide moiety.

6. A molecule according to claim 4, wherein the modified deoxyribonucleotide is N′3-N′5 phosphoroamidate deoxyribonucleotide moiety.

7. A molecule according to claim 1, wherein at least one of the moieties is a ribonucleotide moiety.

8. A molecule according to claim 7, wherein at least one of the moieties is a modified ribonucleotide moiety.

9. A molecule according to claim 8, wherein the modified ribonucleotide is substituted at the 2′ position.

10. A molecule according to claim 9, wherein the substituent at the 2′ position is a C₁ to C₄ alkyl group.

11. A molecule according to claim 10, wherein the alkyl group is methyl.

12. A molecule according to claim 10, wherein the alkyl group is allyl.

13. A molecule according to claim 9, wherein the substituent at the 2′ position is a C₁ to C₄ alkoxy-C₁ to C₄ alkyl group.

14. A molecule according to claim 13, wherein the C₁ to C₄ alkoxy-C₁ to C₄ alkyl group is methoxyethyl.

15. A molecule according to claim 8, wherein the modified ribonucleotide has a methylene bridge between the 2′-oxygen atom and the 4′-carbon atom.

16. A molecule according to claim 1, wherein at least one of the moieties is a peptide nucleic acid moiety.

17. A molecule according to claim 1, wherein at least one of the moieties is a 2′-fluororibonucleotide moiety.

18. A molecule according to claim 1, wherein at least one of the moieties is a morpholino phosphoroamidate nucleotide moiety.

19. A molecule according to claim 1, wherein at least one of the moieties is a tricyclo nucleotide moiety.

20. A molecule according to claim 1, wherein at least one of the moieties is a cyclohexene nucleotide moiety.

21. A molecule according to claim 1, wherein the molecule comprises at least one modified moiety for increased nuclease resistance.

22. A molecule according to claim 21, wherein the nuclease is an exonuclease.

23. A molecule according to claim 22, wherein the molecule comprises at least one modified moiety at the 5′ end.

24. A molecule according to claim 22, wherein the molecule comprises at least two modified moieties at the 5′ end.

25. A molecule according to claim 22, wherein the molecule comprises at least one modified moiety at the 3′ end.

26. A molecule according to claim 22, wherein the molecule comprises at least two modified moieties at the 3′ end.

27. A molecule according to claim 22, wherein the molecule comprises at least one modified moiety at the 5′ end and at least one modified moiety at the 3′ end.

28. A molecule according to claim 22, wherein the molecule comprises at least two modified moieties at the 5′ end and at least two modified moieties at the 3′ end.

29. A molecule according to claim 22, wherein the molecule comprises a nucleotide cap at the 5′ end, the 3′ end or both.

30. A molecule according to claim 22, wherein the molecule comprises an ethylene glycol compound and/or amino linkers at the 5′ end, the 3′ end, or both.

31. A molecule according to claim 1, wherein the nuclease is an endonuclease.

32. A molecule according to claim 31, wherein the molecule comprises at least one modified moiety between the 5′ and 3′ end.

33. A molecule according to claim 31, wherein the molecule comprises an ethylene glycol compound and/or amino linker between the 5′ end and 3′ end.

34. A molecule according to claim 1, wherein all of the moieties are nuclease resistant.

35. A method for inhibiting microRNP activity in a cell, the microRNP comprising a microRNA molecule, the microRNA molecule comprising a sequences of bases complementary of the sequence of bases in a single stranded anti-microRNA molecule, the method comprising introducing into the cell the single-stranded anti-microRNA molecule comprising a sequence of a minimum of ten moieties and a maximum of fifty moieties on a molecular backbone, the molecular backbone comprising backbone units, each moiety comprising a base bonded to a backbone unit, each base forming a Watson-Crick base pair with a complementary base, wherein:
at least ten contiguous bases of the anti-microRNA molecule are complementary to the microRNA, except that up to thirty percent of the bases may be substituted by wobble base pairs, and up to ten percent of the at least ten moieties are addition, deletions, mismatches, or combinations thereof;
no more than fifty percent of the contiguous moieties contain deoxyribonuleotide backbone units; and
the moiety in the molecule at the position corresponding to position 11 of the microRNA is non-complementary.

36. A method according to claim 35, wherein the anti-microRNA is a human anti-microRNA.

37. A method according to claim 35, wherein the anti-microRNA is a mouse anti-microRNA.

38. A method according to claim 35, wherein the anti-microRNA is a rat anti-microRNA.

39. A method according to claim 35, wherein the ant-microRNA is a drosophila microRNA.

40. A method according to claim 35, wherein the anti-microRNA is a C. elegans microRNA.

41. An isolated microRNA molecule comprising a minimum of ten moieties and a maximum of fifty moieties on a molecular backbone, the molecular backbone comprising backbone units, each moiety comprising a base bonded to a backbone unit wherein:
at least ten contiguous bases have the same sequence as a sequence of bases in any one of the microRNA molecules shown in Table 2, except that up to thirty percent of the bases pairs may be wobble base pairs, and up to 10% of the contiguous bases are additions, deletions, mismatches, or combinations thereof; and
no more than fifty percent of the contiguous moieties contain deoxyribonuleotide backbone units.

42. A molecule according to claim 41 having the sequence shown in Table 2.

43. A molecule according to claim 41, wherein the molecule is modified for increased nuclease resistance.

44. A molecule according to claim 41, wherein the moiety at position 11 is an addition, deletion or substitution.

45. An isolated microRNA molecule comprising a minimum of ten moieties and a maximum of fifty moieties on a molecular backbone, the molecular backbone comprising backbone units, each moiety comprising a base bonded to a backbone unit wherein:
at least ten contiguous bases have any one of the microRNA sequences shown in Tables 1, 3 and 4, except that up to thirty percent of the bases pairs may be wobble base pairs, and up to 10% of the contiguous bases are additions, deletions, mismatches, or combinations thereof;
no more than fifty percent of the contiguous moieties contain deoxyribonuleotide backbone units; and
is modified for increased nuclease resistance.

46. A molecule according to claim 45, wherein the molecule is modified for increased nuclease resistance.

47. A molecule according to claim 45, wherein the moiety at position 11 is an addition, deletion, or substitution.

48. An isolated single stranded anti-microRNA molecule comprising a minimum of ten moieties and a maximum of fifty moieties on a molecular backbone, the molecular backbone comprising backbone units, each moiety comprising a base bonded to a backbone unit, each base forming a Watson-Crick base pair with a complementary base wherein:
at least ten contiguous bases have the same sequence as a sequence of bases in any one of the anti-microRNA molecules shown in Tables 1-4, except that up to thirty percent of the bases pairs may be wobble base pairs, and up to 10% of the contiguous bases may be additions, deletions, mismatches, or combinations thereof;
no more than fifty percent of the contiguous moieties contain deoxyribonuleotide backbone units; and
the molecule is capable of inhibiting microRNP activity.

49. A method for inhibiting microRNP activity in a cell, the microRNP comprising a microRNA molecule, the microRNA molecule comprising a sequences of bases complementary of the sequence of bases in a single stranded anti-microRNA molecule, the method comprising introducing into the cell the single-stranded anti-microRNA molecule comprising a sequence of a minimum of ten moieties and a maximum of fifty moieties on a molecular backbone, the molecular backbone comprising backbone units, each moiety comprising a base bonded to a backbone unit, each base forming a Watson-Crick base pair with a complementary base, wherein:
at least ten contiguous bases of the anti-microRNA molecule are complementary to the microRNA, except that up to thirty percent of the bases may be substituted by wobble base pairs, and up to ten percent of the at least ten moieties may be additions, deletions, mismatches, or combinations thereof; and
no more than fifty percent of the contiguous moieties contain deoxyribonuleotide backbone units.


SEQUENCE LISTING (623 sequences)

SEQ ID NO: 1 - RNA, 22 nt, Homo sapiens
aacccguagauccgaacuugug

SEQ ID NO: 2 - RNA, 22 nt, Homo sapiens
agcagcauuguacagggcuaug

SEQ ID NO: 3 - RNA, 22 nt, Homo sapiens
ucaaaugcucagacuccugugg

SEQ ID NO: 4 - RNA, 22 nt, Homo sapiens
aaaagugcuuacagugcaggua

SEQ ID NO: 5 - RNA, 22 nt, Homo sapiens
uaaagugcugacagugcagaua

SEQ ID NO: 6 - RNA, 22 nt, Homo sapiens
agcagcauuguacagggcuauc

SEQ ID NO: 7 - RNA, 22 nt, Homo sapiens
uacccuguagaaccgaauuugu

SEQ ID NO: 8 - RNA, 22 nt, Homo sapiens
ucacagugaaccggucucuuuc

SEQ ID NO: 9 - RNA, 22 nt, Homo sapiens
cagugcaaugaugaaagggcau

SEQ ID NO: 10 - RNA, 22 nt, Homo sapiens
uaccacaggguagaaccacgga

SEQ ID NO: 11 - RNA, 22 nt, Homo sapiens
cccauaaaguagaaagcacuac

SEQ ID NO: 12 - RNA, 22 nt, Homo sapiens
ucgaggagcucacagucuagua

SEQ ID NO: 13 - RNA, 22 nt, Homo sapiens
uuaaugcuaaucgugauagggg

SEQ ID NO: 14 - RNA, 22 nt, Homo sapiens
aacauucaacgcugucggugag

SEQ ID NO: 15 - RNA, 22 nt, Homo sapiens
aacauucauugcugucgguggg

SEQ ID NO: 16 - RNA, 22 nt, Homo sapiens
aacauucaaccugucggugagu

SEQ ID NO: 17 - RNA, 22 nt, Homo sapiens
uuuggcaaugguagaacucaca

SEQ ID NO: 18 - RNA, 22 nt, Homo sapiens
uauggcacugguagaauucacu

SEQ ID NO: 19 - RNA, 22 nt, Homo sapiens
uggacggagaacugauaagggu

SEQ ID NO: 20 - RNA, 22 nt, Homo sapiens
uggagagaaaggcaguuccuga

SEQ ID NO: 21 - RNA, 22 nt, Homo sapiens
caaagaauucuccuuuugggcu

SEQ ID NO: 22 - RNA, 22 nt, Homo sapiens
ucgugucuuguguugcagccgg

SEQ ID NO: 23 - RNA, 22 nt, Homo sapiens
cucccacaugcaggguuugcag

SEQ ID NO: 24 - RNA, 22 nt, Homo sapiens
caucccuugcaugguggagggu

SEQ ID NO: 25 - RNA, 22 nt, Homo sapiens
gugccuacugagcugauaucag

SEQ ID NO: 26 - RNA, 22 nt, Homo sapiens
ugauauguuugauauauuaggu

SEQ ID NO: 27 - RNA, 22 nt, Homo sapiens
caacggaaucccaaaagcagcu

SEQ ID NO: 28 - RNA, 22 nt, Homo sapiens
cugaccuaugaauugacagcca

SEQ ID NO: 29 - RNA, 22 nt, Homo sapiens
aacuggccuacaaagucccagu

SEQ ID NO: 30 - RNA, 22 nt, Homo sapiens
ugggucuuugcgggcaagauga

SEQ ID NO: 31 - RNA, 22 nt, Homo sapiens
uguaacagcaacuccaugugga

SEQ ID NO: 32 - RNA, 22 nt, Homo sapiens
uagcagcacagaaauauuggca

SEQ ID NO: 33 - RNA, 22 nt, Homo sapiens
uagguaguuucauguuguuggg

SEQ ID NO: 34 - RNA, 22 nt, Homo sapiens
uucaccaccuucuccacccagc

SEQ ID NO: 35 - RNA, 22 nt, Homo sapiens
gguccagaggggagauagguuc

SEQ ID NO: 36 - RNA, 22 nt, Homo sapiens
acaguagucugcacauugguua

SEQ ID NO: 37 - RNA, 22 nt, Homo sapiens
cccaguguucagacuaccuguu

SEQ ID NO: 38 - RNA, 22 nt, Homo sapiens
cccaguguuuagacuaucuguu

SEQ ID NO: 39 - RNA, 22 nt, Homo sapiens
uaacacugucugguaacgaugu

SEQ ID NO: 40 - RNA, 22 nt, Homo sapiens
cucuaauacugccugguaauga

SEQ ID NO: 41 - RNA, 22 nt, Homo sapiens
aauacugccggguaaugaugga

SEQ ID NO: 42 - RNA, 22 nt, Homo sapiens
gugaaauguuuaggaccacuag

SEQ ID NO: 43 - RNA, 22 nt, Homo sapiens
uucccuuugucauccuaugccu

SEQ ID NO: 44 - RNA, 22 nt, Homo sapiens
uccuucauuccaccggagucug

SEQ ID NO: 45 - RNA, 22 nt, Homo sapiens
uggaauguaaggaagugugugg

SEQ ID NO: 46 - RNA, 22 nt, Homo sapiens
auaagacgagcaaaaagcuugu

SEQ ID NO: 47 - RNA, 22 nt, Homo sapiens
cugugcgugugacagcggcuga

SEQ ID NO: 48 - RNA, 22 nt, Homo sapiens
uucccuuugucauccuucgccu

SEQ ID NO: 49 - RNA, 22 nt, Homo sapiens
uaacagucuccagucacggcca

SEQ ID NO: 50 - RNA, 22 nt, Homo sapiens
accaucgaccguugauuguacc

SEQ ID NO: 51 - RNA, 22 nt, Homo sapiens
acagcaggcacagacaggcagu

SEQ ID NO: 52 - RNA, 22 nt, Homo sapiens
augaccuaugaauugacagaca

SEQ ID NO: 53 - RNA, 22 nt, Homo sapiens
uaaucucagcuggcaacuguga

SEQ ID NO: 54 - RNA, 22 nt, Homo sapiens
uacugcaucaggaacugauugg

SEQ ID NO: 55 - RNA, 22 nt, Homo sapiens
uugugcuugaucuaaccaugug

SEQ ID NO: 56 - RNA, 22 nt, Homo sapiens
ugauuguccaaacgcaauucuu

SEQ ID NO: 57 - RNA, 22 nt, Homo sapiens
ccacaccguaucugacacuuug

SEQ ID NO: 58 - RNA, 22 nt, Homo sapiens
agcuacauugucugcuggguuu

SEQ ID NO: 59 - RNA, 22 nt, Homo sapiens
agcuacaucuggcuacuggguc

SEQ ID NO: 60 - RNA, 22 nt, Homo sapiens
ugucaguuugucaaauacccca

SEQ ID NO: 61 - RNA, 22 nt, Homo sapiens
caagucacuagugguuccguuu

SEQ ID NO: 62 - RNA, 22 nt, Homo sapiens
aaggagcucacagucuauugag

SEQ ID NO: 63 - RNA, 22 nt, Homo sapiens
cucaaacugugggggcacuuuc

SEQ ID NO: 64 - RNA, 22 nt, Homo sapiens
agggcccccccucaauccuguu

SEQ ID NO: 65 - RNA, 22 nt, Homo sapiens
ugguuuaccgucccacauacau

SEQ ID NO: 66 - RNA, 22 nt, Homo sapiens
cagugcaauaguauugucaaag

SEQ ID NO: 67 - RNA, 22 nt, Homo sapiens
uaagugcuuccauguuuuggug

SEQ ID NO: 68 - RNA, 22 nt, Homo sapiens
uguaaacauccuugacuggaag

SEQ ID NO: 69 - RNA, 22 nt, Homo sapiens
aaaagcuggguugagagggcga

SEQ ID NO: 70 - RNA, 22 nt, Homo sapiens
uaagccagggauuguggguucg

SEQ ID NO: 71 - RNA, 22 nt, Homo sapiens
aaacaugaauugcugcuguauc

SEQ ID NO: 72 - RNA, 22 nt, Homo sapiens
gcacauuacacggucgaccucu

SEQ ID NO: 73 - RNA, 22 nt, Homo sapiens
ccacugccccaggugcugcugg

SEQ ID NO: 74 - RNA, 22 nt, Homo sapiens
cgcauccccuagggcauuggug

SEQ ID NO: 75 - RNA, 22 nt, Homo sapiens
ccucugggcccuuccuccagcc

SEQ ID NO: 76 - RNA, 22 nt, Homo sapiens
cuggcccucucugcccuuccgu

SEQ ID NO: 77 - RNA, 22 nt, Homo sapiens
aacacacccagcuaaccuuuuu

SEQ ID NO: 78 - RNA, 22 nt, Homo sapiens
uggcagugucuuagcugguugu

SEQ ID NO: 79 - RNA, 22 nt, Homo sapiens
aggcagugucauuagcugauug

SEQ ID NO: 80 - RNA, 22 nt, Homo sapiens
aggcaguguaguuagcugauug

SEQ ID NO: 81 - RNA, 22 nt, Homo sapiens
uauugcacuugucccggccugu

SEQ ID NO: 82 - RNA, 22 nt, Homo sapiens
aaagugcuguucgugcagguag

SEQ ID NO: 83 - RNA, 22 nt, Homo sapiens
uucaacggguauuuauugagca

SEQ ID NO: 84 - RNA, 22 nt, Homo sapiens
uuuggcacuagcacauuuuugc

SEQ ID NO: 85 - RNA, 22 nt, Homo sapiens
ugagguaguaaguuguauuguu

SEQ ID NO: 86 - RNA, 22 nt, Mouse
caaagugcuaacagugcaggua

SEQ ID NO: 87 - RNA, 22 nt, Mouse
cccuguagaaccgaauuugugu

SEQ ID NO: 88 - RNA, 22 nt, Mouse
uauggcuuuucauuccuaugug

SEQ ID NO: 89 - RNA, 22 nt, Mouse
ucagugcaucacagaacuuugu

SEQ ID NO: 90 - RNA, 22 nt, Mouse
cuagacugaggcuccuugagga

SEQ ID NO: 91 - RNA, 22 nt, Mouse
uuaaugcuaauugugauagggg

SEQ ID NO: 92 - RNA, 22 nt, Mouse
cccaguguuuagacuaccuguu

SEQ ID NO: 93 - RNA, 22 nt, Mouse
uaauacugccugguaaugauga

SEQ ID NO: 94 - RNA, 22 nt, Mouse
ugaaauguuuaggaccacuaga

SEQ ID NO: 95 - RNA, 22 nt, Mouse
uucccuuugucauccuuugccu

SEQ ID NO: 96 - RNA, 22 nt, Mouse
uacugcaucaggaacugacugg

SEQ ID NO: 97 - RNA, 22 nt, Mouse
uaagucacuagugguuccguuu

SEQ ID NO: 98 - RNA, 22 nt, Mouse
cacuagauugugagcugcugga

SEQ ID NO: 99 - RNA, 22 nt, Mouse
cucaaacuaugggggcacuuuu

SEQ ID NO: 100 - RNA, 22 nt, Mouse
aaagugcuuccacuuugugugc

SEQ ID NO: 101 - RNA, 22 nt, Mouse
caucaaaguggaggcccucucu

SEQ ID NO: 102 - RNA, 22 nt, Mouse
aagugccgccagguuuugagug

SEQ ID NO: 103 - RNA, 22 nt, Mouse
acucaaacugggggcucuuuug

SEQ ID NO: 104 - RNA, 22 nt, Mouse
agugccgcagaguuuguagugu

SEQ ID NO: 105 - RNA, 22 nt, Mouse
aaagugcuucccuuuugugugu

SEQ ID NO: 106 - RNA, 22 nt, Mouse
aaagugcuacuacuuuugaguc

SEQ ID NO: 107 - RNA, 22 nt, Mouse
auguaugugugcaugugcaugu

SEQ ID NO: 108 - RNA, 22 nt, Mouse
ggcagaggagggcuguucuucc

SEQ ID NO: 109 - RNA, 22 nt, Mouse
uaugcaagggcaagcucucuuc

SEQ ID NO: 110 - RNA, 22 nt, Mouse
aggcaagaugcuggcauagcug

SEQ ID NO: 111 - RNA, 22 nt, Mouse
aaacaugaagcgcugcaacacc

SEQ ID NO: 112 - RNA, 22 nt, Mouse
ccuaguaggugcucaguaagug

SEQ ID NO: 113 - RNA, 22 nt, Mouse
ccucugggcccuuccuccaguc

SEQ ID NO: 114 - RNA, 22 nt, Mouse
gcaaagcacagggccugcagag

SEQ ID NO: 115 - RNA, 22 nt, Mouse
gccccugggccuauccuagaac

SEQ ID NO: 116 - RNA, 22 nt, Mouse
uucagcuccuauaugaugccuu

SEQ ID NO: 117 - RNA, 22 nt, Mouse
uccagcaucagugauuuuguug

SEQ ID NO: 118 - RNA, 22 nt, Mouse
ucccuguccuccaggagcucac

SEQ ID NO: 119 - RNA, 22 nt, Mouse
uccgucucaguuacuuuauagc

SEQ ID NO: 120 - RNA, 22 nt, Mouse
ucgaucggucggucggucaguc

SEQ ID NO: 121 - RNA, 22 nt, Mouse
ucucacacagaaaucgcacccg

SEQ ID NO: 122 - RNA, 22 nt, Mouse
ugaucuagccaaagccugacug

SEQ ID NO: 123 - RNA, 22 nt, Mouse
ugcugaccccuaguccagugcu

SEQ ID NO: 124 - RNA, 22 nt, Mouse
ugucugcccgagugccugccuc

SEQ ID NO: 125 - RNA, 22 nt, Mouse
uaggcaguguaauuagcugauu

SEQ ID NO: 126 - RNA, 22 nt, Mouse
uucacaaagcccauacacuuuc

SEQ ID NO: 127 - RNA, 22 nt, Mouse
ucccugaggagcccuuugagcc

SEQ ID NO: 128 - RNA, 22 nt, Mouse
uggaagacuugugauuuuguug

SEQ ID NO: 129 - RNA, 22 nt, Mouse
uauugcacuugucccggccuga

SEQ ID NO: 130 - RNA, 22 nt, Mouse
caaagugcuguucgugcaggua

SEQ ID NO: 131 - RNA, 22 nt, Rat
ccuugaggggcaugaggguagu

SEQ ID NO: 132 - RNA, 22 nt, Rat
guggugugcuaguuacuuuugg

SEQ ID NO: 133 - RNA, 22 nt, Rat
ucaagagcaauaacgaaaaaug

SEQ ID NO: 134 - RNA, 22 nt, Rat
ucacccuuccauaucuagucuc

SEQ ID NO: 135 - RNA, 22 nt, Rat
ucucccuccgugugcccaguau

SEQ ID NO: 136 - RNA, 22 nt, Rat
ugucccucugggucgcccagcu

SEQ ID NO: 137 - RNA, 22 nt, Rat
cagcccugcugucuuaaccucu

SEQ ID NO: 138 - RNA, 22 nt, Rat
agaguaguagguugcauaguac

SEQ ID NO: 139 - RNA, 22 nt, Homo sapiens
uuaucagaaucuccagggguac

SEQ ID NO: 140 - RNA, 22 nt, Homo sapiens
aauccuuggaaccuagguguga

SEQ ID NO: 141 - RNA, 22 nt, Homo sapiens
auugcacgguauccaucuguaa

SEQ ID NO: 142 - RNA, 22 nt, Homo sapiens
cggcggggacggcgauuggucc

SEQ ID NO: 143 - RNA, 22 nt, Homo sapiens
uaaugccccuaaaaauccuuau

SEQ ID NO: 144 - RNA, 22 nt, Homo sapiens
uaacugguugaacaacugaacc

SEQ ID NO: 145 - RNA, 22 nt, Caenorhabditis elegans
ugagguaguagguuguauaguu

SEQ ID NO: 146 - RNA, 22 nt, Caenorhabditis elegans
ucccugagaccucaagugugag

SEQ ID NO: 147 - RNA, 22 nt, Caenorhabditis elegans
uggaauguaaagaaguauguag

SEQ ID NO: 148 - RNA, 22 nt, Caenorhabditis elegans
uaucacagccagcuuugaugug

SEQ ID NO: 149 - RNA, 22 nt, Caenorhabditis elegans
aggcagugugguuagcugguug

SEQ ID NO: 150 - RNA, 22 nt, Caenorhabditis elegans
ucaccggguggaaacuagcagu

SEQ ID NO: 151 - RNA, 22 nt, Caenorhabditis elegans
ucaccgggugaaaauucgcaug

SEQ ID NO: 152 - RNA, 22 nt, Caenorhabditis elegans
ucaccgggugaacacuugcagu

SEQ ID NO: 153 - RNA, 22 nt, Caenorhabditis elegans
ucaccgggagaaaaacuggagu

SEQ ID NO: 154 - RNA, 22 nt, Caenorhabditis elegans
ucaccggguguaaaucagcuug

SEQ ID NO: 155 - RNA, 22 nt, Caenorhabditis elegans
ucaccggguguacaucagcuaa

SEQ ID NO: 156 - RNA, 22 nt, Caenorhabditis elegans
ucaccgggugaaaaaucaccua

SEQ ID NO: 157 - RNA, 22 nt, Caenorhabditis elegans
caccggguuaacaucuacagag

SEQ ID NO: 158 - RNA, 22 nt, Caenorhabditis elegans
uaucacaguuuacuugcugucg

SEQ ID NO: 159 - RNA, 22 nt, Caenorhabditis elegans
ugacuagagacacauucagcuu

SEQ ID NO: 160 - RNA, 22 nt, Caenorhabditis elegans
ugacuagagacacauucagcuu

SEQ ID NO: 161 - RNA, 22 nt, Caenorhabditis elegans
ugucauggagucgcucucuuca

SEQ ID NO: 162 - RNA, 22 nt, Caenorhabditis elegans
ugucauggaggcgcucucuuca

SEQ ID NO: 163 - RNA, 22 nt, Caenorhabditis elegans
ugagguaggcucaguagaugcg

SEQ ID NO: 164 - RNA, 22 nt, Caenorhabditis elegans
aagcaccacgagaagcugcaga

SEQ ID NO: 165 - RNA, 22 nt, Caenorhabditis elegans
ugauaugucugguauucuuggg

SEQ ID NO: 166 - RNA, 22 nt, Caenorhabditis elegans
uacccguagcuccuauccaugu

SEQ ID NO: 167 - RNA, 22 nt, Caenorhabditis elegans
cacccguacauauguuuccgug

SEQ ID NO: 168 - RNA, 22 nt, Caenorhabditis elegans
cacccguacauuuguuuccgug

SEQ ID NO: 169 - RNA, 22 nt, Caenorhabditis elegans
uacccguaaucuucauaauccg

SEQ ID NO: 170 - RNA, 22 nt, Caenorhabditis elegans
uacccguauaaguuucugcuga

SEQ ID NO: 171 - RNA, 22 nt, Caenorhabditis elegans
uacccguaauguuuccgcugag

SEQ ID NO: 172 - RNA, 22 nt, Caenorhabditis elegans
uacccuguagaucgagcugugu

SEQ ID NO: 173 - RNA, 22 nt, Caenorhabditis elegans
ugagaucguucaguacggcaau

SEQ ID NO: 174 - RNA, 22 nt, Caenorhabditis elegans
ucgaaucguuuaucaggaugau

SEQ ID NO: 175 - RNA, 22 nt, Caenorhabditis elegans
uauuaugcacauuuucuaguuc

SEQ ID NO: 176 - RNA, 22 nt, Caenorhabditis elegans
ugacuagaaccguuacucaucu

SEQ ID NO: 177 - RNA, 22 nt, Caenorhabditis elegans
ugauauguaaucuagcuuacag

SEQ ID NO: 178 - RNA, 22 nt, Caenorhabditis elegans
augacacugaagcgaguuggaa

SEQ ID NO: 179 - RNA, 22 nt, Caenorhabditis elegans
uaugacacugaagcguuaccga

SEQ ID NO: 180 - RNA, 22 nt, Caenorhabditis elegans
uaugacacugaagcguaaccga

SEQ ID NO: 181 - RNA, 22 nt, Caenorhabditis elegans
caugacacugauuagggaugug

SEQ ID NO: 182 - RNA, 22 nt, Caenorhabditis elegans
ucacaaccuccuagaaagagua

SEQ ID NO: 183 - RNA, 22 nt, Caenorhabditis elegans
ucgaagacucaaaaguguagac

SEQ ID NO: 184 - RNA, 22 nt, Caenorhabditis elegans
ucgaaaauuaaaaaguguagaa

SEQ ID NO: 185 - RNA, 22 nt, Caenorhabditis elegans
uaauacgucguugguguuucca

SEQ ID NO: 186 - RNA, 22 nt, Caenorhabditis elegans
ugaaagacauggguagugaacg

SEQ ID NO: 187 - RNA, 22 nt, Caenorhabditis elegans
aggcaagauguuggcauagcug

SEQ ID NO: 188 - RNA, 22 nt, Caenorhabditis elegans
uggcaagauguaggcaguucag

SEQ ID NO: 189 - RNA, 22 nt, Caenorhabditis elegans
uggcaagaaauggcagucuaca

SEQ ID NO: 190 - RNA, 22 nt, Caenorhabditis elegans
uuaaagcuaccaaccggcuuca

SEQ ID NO: 191 - RNA, 22 nt, Caenorhabditis elegans
uucguuguugaugaagccuuga

SEQ ID NO: 192 - RNA, 22 nt, Caenorhabditis elegans
uucaucaggccauagcugucca

SEQ ID NO: 193 - RNA, 22 nt, Caenorhabditis elegans
uggaggccugguuguuugugcu

SEQ ID NO: 194 - RNA, 22 nt, Caenorhabditis elegans
auaaagcuagguuaccaaagcu

SEQ ID NO: 195 - RNA, 22 nt, Caenorhabditis elegans
agcuuucgacaugauucugaac

SEQ ID NO: 196 - RNA, 22 nt, Caenorhabditis elegans
ugagaucauuaguugaaagccg

SEQ ID NO: 197 - RNA, 22 nt, Caenorhabditis elegans
ugagaucaucgugaaagcuagu

SEQ ID NO: 198 - RNA, 22 nt, Caenorhabditis elegans
ugagaucaucgugaaagccagu

SEQ ID NO: 199 - RNA, 22 nt, Caenorhabditis elegans
uagcaccauauaaauucaguaa

SEQ ID NO: 200 - RNA, 22 nt, Caenorhabditis elegans
ugagguaguauguaauauugua

SEQ ID NO: 201 - RNA, 22 nt, Caenorhabditis elegans
uacaaaguauuugaaaagucgu

SEQ ID NO: 202 - RNA, 22 nt, Caenorhabditis elegans
uaagugaaugcuuugccacagu

SEQ ID NO: 203 - RNA, 22 nt, Caenorhabditis elegans
gugagcaaaguuucaggugugc

SEQ ID NO: 204 - RNA, 22 nt, Caenorhabditis elegans
ugauauguuguuugaaugcccc

SEQ ID NO: 205 - RNA, 22 nt, Caenorhabditis elegans
uaaggcacgcggugaaugccac

SEQ ID NO: 206 - RNA, 22 nt, Caenorhabditis elegans
aauggcacugcaugaauucacg

SEQ ID NO: 207 - RNA, 22 nt, Caenorhabditis elegans
aaugacacugguuaucuuuucc

SEQ ID NO: 208 - RNA, 22 nt, Caenorhabditis elegans
guauuaguugugcgaccaggag

SEQ ID NO: 209 - RNA, 22 nt, Caenorhabditis elegans
uaagcucgugaucaacaggcag

SEQ ID NO: 210 - RNA, 22 nt, Caenorhabditis elegans
uaaaugcaucuuaacugcggug

SEQ ID NO: 211 - RNA, 22 nt, Caenorhabditis elegans
uugagcaaugcgcaugugcggg

SEQ ID NO: 212 - RNA, 22 nt, Caenorhabditis elegans
uuauugcucgagaauacccuuu

SEQ ID NO: 213 - RNA, 22 nt, Caenorhabditis elegans
uauugcacucuccccggccuga

SEQ ID NO: 214 - RNA, 22 nt, Caenorhabditis elegans
uaauacugucagguaaugacgc

SEQ ID NO: 215 - RNA, 22 nt, Caenorhabditis elegans
ucccugagaauucucgaacagc

SEQ ID NO: 216 - RNA, 22 nt, Caenorhabditis elegans
uuuguacuccgaugccauucag

SEQ ID NO: 217 - RNA, 22 nt, Caenorhabditis elegans
uuuguacuacacauagguacug

SEQ ID NO: 218 - RNA, 22 nt, Caenorhabditis elegans
uuuguacuacacaaaaguacug

SEQ ID NO: 219 - RNA, 22 nt, Caenorhabditis elegans
uacuggcccccaaaucuucgcu

SEQ ID NO: 220 - RNA, 22 nt, Caenorhabditis elegans
ugagguaggugcgagaaaugac

SEQ ID NO: 221 - RNA, 22 nt, Caenorhabditis elegans
uugcguaggccuuugcuucgag

SEQ ID NO: 222 - RNA, 22 nt, Caenorhabditis elegans
cgguacgaucgcggcgggauau

SEQ ID NO: 223 - RNA, 22 nt, Caenorhabditis elegans
ucuuugguuguacaaaguggua

SEQ ID NO: 224 - RNA, 22 nt, Caenorhabditis elegans
auugguccccuccaaguagcuc

SEQ ID NO: 225 - RNA, 22 nt, Caenorhabditis elegans
uuacauguuucggguaggagcu

SEQ ID NO: 226 - RNA, 22 nt, Caenorhabditis elegans
ugacuagagccuauucucuucu

SEQ ID NO: 227 - RNA, 22 nt, Caenorhabditis elegans
uacacgugcacggauaacgcuc

SEQ ID NO: 228 - RNA, 22 nt, Caenorhabditis elegans
ucacaggacuuuugagcguugc

SEQ ID NO: 229 - RNA, 22 nt, Caenorhabditis elegans
ucacagucaacuguuggcaugg

SEQ ID NO: 230 - RNA, 22 nt, Caenorhabditis elegans
uuaaguaguggugccgcucuua

SEQ ID NO: 231 - RNA, 22 nt, Caenorhabditis elegans
uaaguaguagugccgcagguaa

SEQ ID NO: 232 - RNA, 22 nt, Caenorhabditis elegans
cacaccucacuaacacugacca

SEQ ID NO: 233 - RNA, 22 nt, Caenorhabditis elegans
ugcaaaucuuucgcgacuguag

SEQ ID NO: 234 - RNA, 22 nt, Caenorhabditis elegans
uggaaugcauagaagacuguac

SEQ ID NO: 235 - RNA, 22 nt, Caenorhabditis elegans
gaguaucaggaguacccaguga

SEQ ID NO: 236 - RNA, 22 nt, Caenorhabditis elegans
gguuuugagaggaauccuuuua

SEQ ID NO: 237 - RNA, 22 nt, Caenorhabditis elegans
aguaaaucucauccuaaucugg

SEQ ID NO: 238 - RNA, 22 nt, Caenorhabditis elegans
gugaugucgaacucuuguagga

SEQ ID NO: 239 - RNA, 22 nt, Caenorhabditis elegans
uagcuuuuuaguuuucacggug

SEQ ID NO: 240 - RNA, 22 nt, Caenorhabditis elegans
guuucucgauguuuucugauac

SEQ ID NO: 241 - RNA, 22 nt, Caenorhabditis elegans
ggcgggugguuguuguuauggg

SEQ ID NO: 242 - RNA, 22 nt, Caenorhabditis elegans
ugagggaggaagggugguauuu

SEQ ID NO: 243 - RNA, 22 nt, Caenorhabditis elegans
aggcaagacuuuggcaaagcuu

SEQ ID NO: 244 - RNA, 22 nt, Caenorhabditis elegans
cccgugaagugucugcugcaau

SEQ ID NO: 245 - RNA, 22 nt, Caenorhabditis elegans
ggcaagaauuagaagcaguuug

SEQ ID NO: 246 - RNA, 22 nt, Caenorhabditis elegans
ggcaagacucuggcaaaacuug

SEQ ID NO: 247 - RNA, 22 nt, Caenorhabditis elegans
ggcaugauguagcaguggagau

SEQ ID NO: 248 - RNA, 22 nt, Caenorhabditis elegans
ucgccgggugggaaagcauucg

SEQ ID NO: 249 - RNA, 22 nt, Caenorhabditis elegans
uguaggcauggguguuuggaag

SEQ ID NO: 250 - RNA, 22 nt, Caenorhabditis elegans
ugcccguacugugucggcugcu

SEQ ID NO: 251 - RNA, 22 nt, Drosophila melanogaster
guuaauggcacuggaagaauuc

SEQ ID NO: 252 - RNA, 22 nt, Drosophila melanogaster
uggacggagaacugauaagggc

SEQ ID NO: 253 - RNA, 22 nt, Drosophila melanogaster
uuuugugaccgacacuaacggg

SEQ ID NO: 254 - RNA, 22 nt, Drosophila melanogaster
ucagguaccugaaguagcgcgc

SEQ ID NO: 255 - RNA, 22 nt, Drosophila melanogaster
cauugcacuugucccggccuau

SEQ ID NO: 256 - RNA, 22 nt, Drosophila melanogaster
ugauuguccaaacgcaauucuu

SEQ ID NO: 257 - RNA, 22 nt, Drosophila melanogaster
uaggaacuucauaccgugcucu

SEQ ID NO: 258 - RNA, 22 nt, Drosophila melanogaster
uaaaugcacuaucugguacgac

SEQ ID NO: 259 - RNA, 22 nt, Drosophila melanogaster
ucggugggacuuucguccguuu

SEQ ID NO: 260 - RNA, 22 nt, Drosophila melanogaster
uugguccccuucaaccagcugu

SEQ ID NO: 261 - RNA, 22 nt, Drosophila melanogaster
ugacuagauccacacucauuaa

SEQ ID NO: 262 - RNA, 22 nt, Drosophila melanogaster
aggugcauuguagucgcauugu

SEQ ID NO: 263 - RNA, 22 nt, Drosophila melanogaster
uguauuuacguugcauaugaaa

SEQ ID NO: 264 - RNA, 22 nt, Drosophila melanogaster
ugucauggaauugcucucuuug

SEQ ID NO: 265 - RNA, 22 nt, Drosophila melanogaster
aaucuagccucuacuaggcuuu

SEQ ID NO: 266 - RNA, 22 nt, Drosophila melanogaster
uaaauaucagcugguaauucug

SEQ ID NO: 267 - RNA, 22 nt, Drosophila melanogaster
ugaagucagcaacuugauucca

SEQ ID NO: 268 - RNA, 22 nt, Drosophila melanogaster
uggcagugugguuagcugguug

SEQ ID NO: 269 - RNA, 22 nt, Drosophila melanogaster
uaaggcacgcggugaaugccaa

SEQ ID NO: 270 - RNA, 22 nt, Drosophila melanogaster
uaaagcuagauuaccaaagcau

SEQ ID NO: 271 - RNA, 22 nt, Drosophila melanogaster
uaggaacuuaauaccgugcucu

SEQ ID NO: 272 - RNA, 22 nt, Drosophila melanogaster
uugugcgugugacagcggcuau

SEQ ID NO: 273 - RNA, 22 nt, Drosophila melanogaster
uagcaccauucgaaaucagugc

SEQ ID NO: 274 - RNA, 22 nt, Drosophila melanogaster
aacccguaaauccgaacuugug

SEQ ID NO: 275 - RNA, 22 nt, Drosophila melanogaster
aauugcacuagucccggccugc

SEQ ID NO: 276 - RNA, 22 nt, Drosophila melanogaster
ugacuagaccgaacacucgugc

SEQ ID NO: 277 - RNA, 22 nt, Drosophila melanogaster
uguguugaaaaucguuugcacg

SEQ ID NO: 278 - RNA, 22 nt, Drosophila melanogaster
uugagcaaaauuucaggugugu

SEQ ID NO: 279 - RNA, 22 nt, Drosophila melanogaster
cuuggcacugggagaauucaca

SEQ ID NO: 280 - RNA, 22 nt, Drosophila melanogaster
uuucaugucgauuucauuucau

SEQ ID NO: 281 - RNA, 22 nt, Drosophila melanogaster
uaaauauuuaaguggagccugc

SEQ ID NO: 282 - RNA, 22 nt, Drosophila melanogaster
ugagaucauuuugaaagcugau

SEQ ID NO: 283 - RNA, 22 nt, Drosophila melanogaster
uuuagguuucacaggaaacugg

SEQ ID NO: 284 - RNA, 22 nt, Drosophila melanogaster
uggcaagaugucggaauagcug

SEQ ID NO: 285 - RNA, 22 nt, Drosophila melanogaster
uaaucucaauuuguaaauguga

SEQ ID NO: 286 - RNA, 22 nt, Drosophila melanogaster
auuguacuucaucaggugcucu

SEQ ID NO: 287 - RNA, 22 nt, Drosophila melanogaster
ucuuugguauucuagcuguaga

SEQ ID NO: 288 - RNA, 22 nt, Drosophila melanogaster
ucagguacuuagugacucucaa

SEQ ID NO: 289 - RNA, 22 nt, Drosophila melanogaster
ucuuuggugauuuuagcuguau

SEQ ID NO: 290 - RNA, 22 nt, Drosophila melanogaster
ucccugagacccuaacuuguga

SEQ ID NO: 291 - RNA, 22 nt, Drosophila melanogaster
ucacaaccuccuugagugagcg

SEQ ID NO: 292 - RNA, 22 nt, Drosophila melanogaster
aaucacaggauuauacugugag

SEQ ID NO: 293 - RNA, 22 nt, Drosophila melanogaster
uggcaagaugucggcauagcug

SEQ ID NO: 294 - RNA, 22 nt, Drosophila melanogaster
gcacuggguaaaguuuguccua

SEQ ID NO: 295 - RNA, 22 nt, Drosophila melanogaster
uauugcacacuucccggccuuu

SEQ ID NO: 296 - RNA, 22 nt, Drosophila melanogaster
uauugcacauucaccggccuga

SEQ ID NO: 297 - RNA, 22 nt, Drosophila melanogaster
uauugcacuugagacggccuga

SEQ ID NO: 298 - RNA, 22 nt, Drosophila melanogaster
uauugcacuuuucacagcccga

SEQ ID NO: 299 - RNA, 21 nt, Drosophila melanogaster
uauucgagccaauaaguucgg

SEQ ID NO: 300 - RNA, 22 nt, Drosophila melanogaster
uuuugauuguugcucagaaagc

SEQ ID NO: 301 - RNA, 22 nt, Drosophila melanogaster
ugucuuuuuccgcuuacuggcg

SEQ ID NO: 302 - RNA, 22 nt, Drosophila melanogaster
ugaacacagcuggugguaucca

SEQ ID NO: 303 - RNA, 22 nt, Drosophila melanogaster
ucacugggcuuuguuuaucuca

SEQ ID NO: 304 - RNA, 22 nt, Drosophila melanogaster
uaucacagccagcuuugauggg

SEQ ID NO: 305 - RNA, 22 nt, Drosophila melanogaster
acguauacugaauguauccuga

SEQ ID NO: 306 - RNA, 22 nt, Drosophila melanogaster
cgguauaccuucaguauacgua

SEQ ID NO: 307 - RNA, 22 nt, Artificial
  Anti-microRNA molecule
cacaaguucggaucuacggguu

SEQ ID NO: 308 - RNA, 22 nt, Artificial
  Anti-microRNA molecule
cauagcccuguacaaugcugcu

SEQ ID NO: 309 - RNA, 22 nt, Artificial
  anti-microRNA molecule
ccacaggagucugagcauuuga

SEQ ID NO: 310 - RNA, 22 nt, Artificial
  anti-microRNA molecule
uaccugcacuguaagcacuuuu

SEQ ID NO: 311 - RNA, 22 nt, Artificial
  anti-microRNA molecule
uaucugcacugucagcacuuua

SEQ ID NO: 312 - RNA, 22 nt, Artificial
  anti-microRNA molecule
gauagcccuguacaaugcugcu

SEQ ID NO: 313 - RNA, 22 nt, Artificial
  anti-microRNA molecule
acaaauucgguucuacagggua

SEQ ID NO: 314 - RNA, 22 nt, Artificial
  anti-microRNA molecule
gaaagagaccgguucacuguga

SEQ ID NO: 315 - RNA, 22 nt, Artificial
  anti-microRNA molecule
augcccuuucaucauugcacug

SEQ ID NO: 316 - RNA, 22 nt, Artificial
  anti-microRNA molecule
uccgugguucuacccuguggua

SEQ ID NO: 317 - RNA, 22 nt, Artificial
  anti-microRNA molecule
guagugcuuucuacuuuauggg

SEQ ID NO: 318 - RNA, 22 nt, Artificial
  anti-microRNA molecule
uacuagacugugagcuccucga

SEQ ID NO: 319 - RNA, 22 nt, Artificial
  anti-microRNA molecule
ccccuaucacgauuagcauuaa

SEQ ID NO: 320 - RNA, 22 nt, Artificial
  anti-microRNA molecule
cucaccgacagcguugaauguu

SEQ ID NO: 321 - RNA, 22 nt, Artificial
  anti-microRNA molecule
cccaccgacagcaaugaauguu

SEQ ID NO: 322 - RNA, 22 nt, Artificial
  anti-microRNA molecule
acucaccgacagguugaauguu

SEQ ID NO: 323 - RNA, 22 nt, Artificial
  anti-microRNA molecule
ugugaguucuaccauugccaaa

SEQ ID NO: 324 - RNA, 22 nt, Artificial
  anti-microRNA molecule
agugaauucuaccagugccaua

SEQ ID NO: 325 - RNA, 22 nt, Artificial
  anti-microRNA molecule
acccuuaucaguucuccgucca

SEQ ID NO: 326 - RNA, 22 nt, Artificial
  anti-microRNA molecule
ucaggaacugccuuucucucca

SEQ ID NO: 327 - RNA, 22 nt, Artificial
  anti-microRNA molecule
agcccaaaaggagaauucuuug

SEQ ID NO: 328 - RNA, 22 nt, Artificial
  anti-microRNA molecule
ccggcugcaacacaagacacga

SEQ ID NO: 329 - RNA, 22 nt, Artificial
  anti-microRNA molecule
cugcaaacccugcaugugggag

SEQ ID NO: 330 - RNA, 22 nt, Artificial
  anti-microRNA molecule
acccuccaccaugcaagggaug

SEQ ID NO: 331 - RNA, 22 nt, Artificial
  anti-microRNA molecule
cugauaucagcucaguaggcac

SEQ ID NO: 332 - RNA, 22 nt, Artificial
  anti-microRNA molecule
accuaauauaucaaacauauca

SEQ ID NO: 333 - RNA, 22 nt, Artificial
  anti-microRNA molecule
agcugcuuuugggauuccguug

SEQ ID NO: 334 - RNA, 22 nt, Artificial
  anti-microRNA molecule
uggcugucaauucauaggucag

SEQ ID NO: 335 - RNA, 22 nt, Artificial
  anti-microRNA molecule
acugggacuuuguaggccaguu

SEQ ID NO: 336 - RNA, 22 nt, Artificial
  anti-microRNA molecule
ucaucuugcccgcaaagaccca

SEQ ID NO: 337 - RNA, 22 nt, Artificial
  anti-microRNA molecule
uccacauggaguugcuguuaca

SEQ ID NO: 338 - RNA, 22 nt, Artificial
  anti-microRNA molecule
ugccaauauuucugugcugcua

SEQ ID NO: 339 - RNA, 22 nt, Artificial
  anti-microRNA molecule
cccaacaacaugaaacuaccua

SEQ ID NO: 340 - RNA, 22 nt, Artificial
  anti-microRNA molecule
gcuggguggagaagguggugaa

SEQ ID NO: 341 - RNA, 22 nt, Artificial
  anti-microRNA molecule
gaaccuaucuccccucuggacc

SEQ ID NO: 342 - RNA, 22 nt, Artificial
  anti-microRNA molecule
uaaccaaugugcagacuacugu

SEQ ID NO: 343 - RNA, 22 nt, Artificial
  anti-microRNA molecule
aacagguagucugaacacuggg

SEQ ID NO: 344 - RNA, 22 nt, Artificial
  anti-microRNA molecule
aacagauagucuaaacacuggg

SEQ ID NO: 345 - RNA, 22 nt, Artificial
  anti-microRNA molecule
acaucguuaccagacaguguua

SEQ ID NO: 346 - RNA, 22 nt, Artificial
  anti-microRNA molecule
ucauuaccaggcaguauuagag

SEQ ID NO: 347 - RNA, 22 nt, Artificial
  anti-microRNA molecule
uccaucauuacccggcaguauu

SEQ ID NO: 348 - RNA, 22 nt, Artificial
  anti-microRNA molecule
cuagugguccuaaacauuucac

SEQ ID NO: 349 - RNA, 22 nt, Artificial
  anti-microRNA molecule
aggcauaggaugacaaagggaa

SEQ ID NO: 350 - RNA, 22 nt, Artificial
  anti-microRNA molecule
cagacuccgguggaaugaagga

SEQ ID NO: 351 - RNA, 22 nt, Artificial
  anti-microRNA molecule
ccacacacuuccuuacauucca

SEQ ID NO: 352 - RNA, 22 nt, Artificial
  anti-microRNA molecule
acaagcuuuuugcucgucuuau

SEQ ID NO: 353 - RNA, 22 nt, Artificial
  anti-microRNA molecule
ucagccgcugucacacgcacag

SEQ ID NO: 354 - RNA, 22 nt, Artificial
  anti-microRNA molecule
aggcgaaggaugacaaagggaa

SEQ ID NO: 355 - RNA, 22 nt, Artificial
  anti-microRNA molecule
uggccgugacuggagacuguua

SEQ ID NO: 356 - RNA, 22 nt, Artificial
  anti-microRNA molecule
gguacaaucaacggucgauggu

SEQ ID NO: 357 - RNA, 22 nt, Artificial
  anti-microRNA molecule
acugccugucugugccugcugu

SEQ ID NO: 358 - RNA, 22 nt, Artificial
  anti-microRNA molecule
ugucugucaauucauaggucau

SEQ ID NO: 359 - RNA, 22 nt, Artificial
  anti-microRNA molecule
ucacaguugccagcugagauua

SEQ ID NO: 360 - RNA, 22 nt, Artificial
  anti-microRNA molecule
ccaaucaguuccugaugcagua

SEQ ID NO: 361 - RNA, 22 nt, Artificial
  anti-microRNA molecule
cacaugguuagaucaagcacaa

SEQ ID NO: 362 - RNA, 22 nt, Artificial
  anti-microRNA molecule
aagaauugcguuuggacaauca

SEQ ID NO: 363 - RNA, 22 nt, Artificial
  anti-microRNA molecule
caaagugucagauacggugugg

SEQ ID NO: 364 - RNA, 22 nt, Artificial
  anti-microRNA molecule
aaacccagcagacaauguagcu

SEQ ID NO: 365 - RNA, 22 nt, Artificial
  anti-microRNA molecule
gacccaguagccagauguagcu

SEQ ID NO: 366 - RNA, 22 nt, Artificial
  anti-microRNA molecule
ugggguauuugacaaacugaca

SEQ ID NO: 367 - RNA, 22 nt, Artificial
  anti-microRNA molecule
aaacggaaccacuagugacuug

SEQ ID NO: 368 - RNA, 22 nt, Artificial
  anti-microRNA molecule
cucaauagacugugagcuccuu

SEQ ID NO: 369 - RNA, 22 nt, Artificial
  anti-microRNA molecule
gaaagugcccccacaguuugag

SEQ ID NO: 370 - RNA, 22 nt, Artificial
  anti-microRNA molecule
aacaggauugagggggggcccu

SEQ ID NO: 371 - RNA, 22 nt, Artificial
  anti-microRNA molecule
auguaugugggacgguaaacca

SEQ ID NO: 372 - RNA, 22 nt, Artificial
  anti-microRNA molecule
cuuugacaauacuauugcacug

SEQ ID NO: 373 - RNA, 22 nt, Artificial
  anti-microRNA molecule
caccaaaacauggaagcacuua

SEQ ID NO: 374 - RNA, 22 nt, Artificial
  anti-microRNA molecule
cuuccagucaaggauguuuaca

SEQ ID NO: 375 - RNA, 22 nt, Artificial
  anti-microRNA molecule
ucgcccucucaacccagcuuuu

SEQ ID NO: 376 - RNA, 22 nt, Artificial
  anti-microRNA molecule
cgaacccacaaucccuggcuua

SEQ ID NO: 377 - RNA, 22 nt, Artificial
  anti-microRNA molecule
gauacagcagcaauucauguuu

SEQ ID NO: 378 - RNA, 22 nt, Artificial
  anti-microRNA molecule
agaggucgaccguguaaugugc

SEQ ID NO: 379 - RNA, 22 nt, Artificial
  anti-microRNA molecule
ccagcagcaccuggggcagugg

SEQ ID NO: 380 - RNA, 22 nt, Artificial
  anti-microRNA molecule
caccaaugcccuaggggaugcg

SEQ ID NO: 381 - RNA, 22 nt, Artificial
  anti-microRNA molecule
ggcuggaggaagggcccagagg

SEQ ID NO: 382 - RNA, 22 nt, Artificial
  anti-microRNA molecule
acggaagggcagagagggccag

SEQ ID NO: 383 - RNA, 22 nt, Artificial
  anti-microRNA molecule
aaaaagguuagcuggguguguu

SEQ ID NO: 384 - RNA, 22 nt, Artificial
  anti-microRNA molecule
acaaccagcuaagacacugcca

SEQ ID NO: 385 - RNA, 22 nt, Artificial
  anti-microRNA molecule
caaucagcuaaugacacugccu

SEQ ID NO: 386 - RNA, 22 nt, Artificial
  anti-microRNA molecule
caaucagcuaacuacacugccu

SEQ ID NO: 387 - RNA, 22 nt, Artificial
  anti-microRNA molecule
acaggccgggacaagugcaaua

SEQ ID NO: 388 - RNA, 22 nt, Artificial
  anti-microRNA molecule
cuaccugcacgaacagcacuuu

SEQ ID NO: 389 - RNA, 22 nt, Artificial
  anti-microRNA molecule
ugcucaauaaauacccguugaa

SEQ ID NO: 390 - RNA, 22 nt, Artificial
  anti-microRNA molecule
gcaaaaaugugcuagugccaaa

SEQ ID NO: 391 - RNA, 22 nt, Artificial
  anti-microRNA molecule
aacaauacaacuuacuaccuca

SEQ ID NO: 392 - RNA, 22 nt, Artificial
  anti-microRNA molecule
uaccugcacuguuagcacuuug

SEQ ID NO: 393 - RNA, 22 nt, Artificial
  anti-microRNA molecule
acacaaauucgguucuacaggg

SEQ ID NO: 394 - RNA, 22 nt, Artificial
  anti-microRNA molecule
cacauaggaaugaaaagccaua

SEQ ID NO: 395 - RNA, 22 nt, Artificial
  anti-microRNA molecule
acaaaguucugugaugcacuga

SEQ ID NO: 396 - RNA, 22 nt, Artificial
  anti-microRNA molecule
uccucaaggagccucagucuag

SEQ ID NO: 397 - RNA, 22 nt, Artificial
  anti-microRNA molecule
ccccuaucacaauuagcauuaa

SEQ ID NO: 398 - RNA, 22 nt, Artificial
  anti-microRNA molecule
aacagguagucuaaacacuggg

SEQ ID NO: 399 - RNA, 22 nt, Artificial
  anti-microRNA molecule
ucaucauuaccaggcaguauua

SEQ ID NO: 400 - RNA, 22 nt, Artificial
  anti-microRNA molecule
ucuagugguccuaaacauuuca

SEQ ID NO: 401 - RNA, 22 nt, Artificial
  anti-microRNA molecule
aggcaaaggaugacaaagggaa

SEQ ID NO: 402 - RNA, 22 nt, Artificial
  anti-microRNA molecule
ccagucaguuccugaugcagua

SEQ ID NO: 403 - RNA, 22 nt, Artificial
  anti-microRNA molecule
aaacggaaccacuagugacuua

SEQ ID NO: 404 - RNA, 22 nt, Artificial
  anti-microRNA molecule
uccagcagcucacaaucuagug

SEQ ID NO: 405 - RNA, 22 nt, Artificial
  anti-microRNA molecule
aaaagugcccccauaguuugag

SEQ ID NO: 406 - RNA, 22 nt, Artificial
  anti-microRNA molecule
gcacacaaaguggaagcacuuu

SEQ ID NO: 407 - RNA, 22 nt, Artificial
  anti-microRNA molecule
agagagggccuccacuuugaug

SEQ ID NO: 408 - RNA, 22 nt, Artificial
  anti-microRNA molecule
cacucaaaaccuggcggcacuu

SEQ ID NO: 409 - RNA, 22 nt, Artificial
  anti-microRNA molecule
caaaagagcccccaguuugagu

SEQ ID NO: 410 - RNA, 22 nt, Artificial
  anti-microRNA molecule
acacuacaaacucugcggcacu

SEQ ID NO: 411 - RNA, 22 nt, Artificial
  anti-microRNA molecule
acacacaaaagggaagcacuuu

SEQ ID NO: 412 - RNA, 22 nt, Artificial
  anti-microRNA molecule
gacucaaaaguaguagcacuuu

SEQ ID NO: 413 - RNA, 22 nt, Artificial
  anti-microRNA molecule
acaugcacaugcacacauacau

SEQ ID NO: 414 - RNA, 22 nt, Artificial
  anti-microRNA molecule
ggaagaacagcccuccucugcc

SEQ ID NO: 415 - RNA, 22 nt, Artificial
  anti-microRNA molecule
gaagagagcuugcccuugcaua

SEQ ID NO: 416 - RNA, 22 nt, Artificial
  anti-microRNA molecule
cagcuaugccagcaucuugccu

SEQ ID NO: 417 - RNA, 22 nt, Artificial
  anti-microRNA molecule
gguguugcagcgcuucauguuu

SEQ ID NO: 418 - RNA, 22 nt, Artificial
  anti-microRNA molecule
cacuuacugagcaccuacuagg

SEQ ID NO: 419 - RNA, 22 nt, Artificial
  anti-microRNA molecule
gacuggaggaagggcccagagg

SEQ ID NO: 420 - RNA, 22 nt, Artificial
  anti-microRNA molecule
cucugcaggcccugugcuuugc

SEQ ID NO: 421 - RNA, 22 nt, Artificial
  anti-microRNA molecule
guucuaggauaggcccaggggc

SEQ ID NO: 422 - RNA, 22 nt, Artificial
  anti-microRNA molecule
aaggcaucauauaggagcugaa

SEQ ID NO: 423 - RNA, 22 nt, Artificial
  anti-microRNA molecule
caacaaaaucacugaugcugga

SEQ ID NO: 424 - RNA, 22 nt, Artificial
  anti-microRNA molecule
gugagcuccuggaggacaggga

SEQ ID NO: 425 - RNA, 22 nt, Artificial
  anti-microRNA molecule
gcuauaaaguaacugagacgga

SEQ ID NO: 426 - RNA, 22 nt, Artificial
  anti-microRNA molecule
gacugaccgaccgaccgaucga

SEQ ID NO: 427 - RNA, 22 nt, Artificial
  anti-microRNA molecule
cgggugcgauuucugugugaga

SEQ ID NO: 428 - RNA, 22 nt, Artificial
  anti-microRNA molecule
cagucaggcuuuggcuagauca

SEQ ID NO: 429 - RNA, 22 nt, Artificial
  anti-microRNA molecule
agcacuggacuaggggucagca

SEQ ID NO: 430 - RNA, 22 nt, Artificial
  anti-microRNA molecule
gaggcaggcacucgggcagaca

SEQ ID NO: 431 - RNA, 22 nt, Artificial
  anti-microRNA molecule
aaucagcuaauuacacugccua

SEQ ID NO: 432 - RNA, 22 nt, Artificial
  anti-microRNA molecule
gaaaguguaugggcuuugugaa

SEQ ID NO: 433 - RNA, 22 nt, Artificial
  anti-microRNA molecule
ggcucaaagggcuccucaggga

SEQ ID NO: 434 - RNA, 22 nt, Artificial
  anti-microRNA molecule
caacaaaaucacaagucuucca

SEQ ID NO: 435 - RNA, 22 nt, Artificial
  anti-microRNA molecule
ucaggccgggacaagugcaaua

SEQ ID NO: 436 - RNA, 22 nt, Artificial
  anti-microRNA  molecule
uaccugcacgaacagcacuuug

SEQ ID NO: 437 - RNA, 22 nt, Artificial
  anti-microRNA molecule
acuacccucaugccccucaagg

SEQ ID NO: 438 - RNA, 22 nt, Artificial
  anti-microRNA molecule
ccaaaaguaacuagcacaccac

SEQ ID NO: 439 - RNA, 22 nt, Artificial
  anti-microRNA molecule
cauuuuucguuauugcucuuga

SEQ ID NO: 440 - RNA, 22 nt, Artificial
  anti-microRNA molecule
gagacuagauauggaaggguga

SEQ ID NO: 441 - RNA, 22 nt, Artificial
  anti-microRNA molecule
auacugggcacacggagggaga

SEQ ID NO: 442 - RNA, 22 nt, Artificial
  anti-microRNA molecule
agcugggcgacccagagggaca

SEQ ID NO: 443 - RNA, 22 nt, Artificial
  anti-microRNA molecule
agagguuaagacagcagggcug

SEQ ID NO: 444 - RNA, 22 nt, Artificial
  anti-microRNA molecule
guacuaugcaaccuacuacucu

SEQ ID NO: 445 - RNA, 22 nt, Artificial
  anti-microRNA molecule
guaccccuggagauucugauaa

SEQ ID NO: 446 - RNA, 22 nt, Artificial
  anti-microRNA molecule
ucacaccuagguuccaaggauu

SEQ ID NO: 447 - RNA, 22 nt, Artificial
  anti-microRNA molecule
uuacagauggauaccgugcaau

SEQ ID NO: 448 - RNA, 22 nt, Artificial
  anti-microRNA molecule
ggaccaaucgccguccccgccg

SEQ ID NO: 449 - RNA, 22 nt, Artificial
  anti-microRNA molecule
auaaggauuuuuaggggcauua

SEQ ID NO: 450 - RNA, 22 nt, Artificial
  anti-microRNA molecule
gguucaguuguucaaccaguua

SEQ ID NO: 451 - RNA, 22 nt, Artificial
  anti-microRNA molecule
aacuauacaaccuacuaccuca

SEQ ID NO: 452 - RNA, 22 nt, Artificial
  anti-microRNA molecule
cucacacuugaggucucaggga

SEQ ID NO: 453 - RNA, 22 nt, Artificial
  anti-microRNA molecule
cuacauacuucuuuacauucca

SEQ ID NO: 454 - RNA, 22 nt, Artificial
  anti-microRNA molecule
cacaucaaagcuggcugugaua

SEQ ID NO: 455 - RNA, 22 nt, Artificial
  anti-microRNA molecule
caaccagcuaaccacacugccu

SEQ ID NO: 456 - RNA, 22 nt, Artificial
  anti-microRNA molecule
acugcuaguuuccacccgguga

SEQ ID NO: 457 - RNA, 22 nt, Artificial
  anti-microRNA molecule
caugcgaauuuucacccgguga

SEQ ID NO: 458 - RNA, 22 nt, Artificial
  anti-microRNA molecule
acugcaaguguucacccgguga

SEQ ID NO: 459 - RNA, 22 nt, Artificial
  anti-microRNA molecule
acuccaguuuuucucccgguga

SEQ ID NO: 460 - RNA, 22 nt, Artificial
  anti-microRNA molecule
caagcugauuuacacccgguga

SEQ ID NO: 461 - RNA, 22 nt, Artificial
  anti-microRNA molecule
uuagcugauguacacccgguga

SEQ ID NO: 462 - RNA, 22 nt, Artificial
  anti-microRNA molecule
uaggugauuuuucacccgguga

SEQ ID NO: 463 - RNA, 22 nt, Artificial
  anti-microRNA molecule
cucuguagauguuaacccggug

SEQ ID NO: 464 - RNA, 22 nt, Artificial
  anti-microRNA molecule
cgacagcaaguaaacugugaua

SEQ ID NO: 465 - RNA, 22 nt, Artificial
  anti-microRNA molecule
aagcugaaugugucucuaguca

SEQ ID NO: 466 - RNA, 22 nt, Artificial
  anti-microRNA molecule
aagcugaaugugucucuaguca

SEQ ID NO: 467 - RNA, 22 nt, Artificial
  anti-microRNA molecule
ugaagagagcgacuccaugaca

SEQ ID NO: 468 - RNA, 22 nt, Artificial
  anti-microRNA molecule
ugaagagagcgccuccaugaca

SEQ ID NO: 469 - RNA, 22 nt, Artificial
  anti-microRNA molecule
cgcaucuacugagccuaccuca

SEQ ID NO: 470 - RNA, 22 nt, Artificial
  anti-microRNA molecule
ucugcagcuucucguggugcuu

SEQ ID NO: 471 - RNA, 22 nt, Artificial
  anti-microRNA molecule
cccaagaauaccagacauauca

SEQ ID NO: 472 - RNA, 22 nt, Artificial
  anti-microRNA molecule
acauggauaggagcuacgggua

SEQ ID NO: 473 - RNA, 22 nt, Artificial
  anti-microRNA molecule
cacggaaacauauguacgggug

SEQ ID NO: 474 - RNA, 22 nt, Artificial
  anti-microRNA molecule
cacggaaacaaauguacgggug

SEQ ID NO: 475 - RNA, 22 nt, Artificial
  anti-microRNA molecule
cggauuaugaagauuacgggua

SEQ ID NO: 476 - RNA, 22 nt, Artificial
  anti-microRNA molecule
ucagcagaaacuuauacgggua

SEQ ID NO: 477 - RNA, 22 nt, Artificial
  anti-microRNA molecule
cucagcggaaacauuacgggua

SEQ ID NO: 478 - RNA, 22 nt, Artificial
  anti-microRNA molecule
acacagcucgaucuacagggua

SEQ ID NO: 479 - RNA, 22 nt, Artificial
  anti-microRNA molecule
auugccguacugaacgaucuca

SEQ ID NO: 480 - RNA, 22 nt, Artificial
  anti-microRNA molecule
aucauccugauaaacgauucga

SEQ ID NO: 481 - RNA, 22 nt, Artificial
  anti-microRNA molecule
gaacuagaaaaugugcauaaua

SEQ ID NO: 482 - RNA, 22 nt, Artificial
  anti-microRNA molecule
agaugaguaacgguucuaguca

SEQ ID NO: 483 - RNA, 22 nt, Artificial
  anti-microRNA molecule
cuguaagcuagauuacauauca

SEQ ID NO: 484 - RNA, 22 nt, Artificial
  anti-microRNA molecule
uuccaacucgcuucagugucau

SEQ ID NO: 485 - RNA, 22 nt, Artificial
  anti-microRNA molecule
ucgguaacgcuucagugucaua

SEQ ID NO: 486 - RNA, 22 nt, Artificial
  anti-microRNA molecule
ucgguuacgcuucagugucaua

SEQ ID NO: 487 - RNA, 22 nt, Artificial
  anti-microRNA molecule
cacaucccuaaucagugucaug

SEQ ID NO: 488 - RNA, 22 nt, Artificial
  anti-microRNA molecule
uacucuuucuaggagguuguga

SEQ ID NO: 489 - RNA, 22 nt, Artificial
  anti-microRNA molecule
gucuacacuuuugagucuucga

SEQ ID NO: 490 - RNA, 22 nt, Artificial
  anti-microRNA molecule
uucuacacuuuuuaauuuucga

SEQ ID NO: 491 - RNA, 22 nt, Artificial
  anti-microRNA molecule
uggaaacaccaacgacguauua

SEQ ID NO: 492 - RNA, 22 nt, Artificial
  anti-microRNA molecule
cguucacuacccaugucuuuca

SEQ ID NO: 493 - RNA, 22 nt, Artificial
  anti-microRNA molecule
cagcuaugccaacaucuugccu

SEQ ID NO: 494 - RNA, 22 nt, Artificial
  anti-microRNA molecule
cugaacugccuacaucuugcca

SEQ ID NO: 495 - RNA, 22 nt, Artificial
  anti-microRNA molecule
uguagacugccauuucuugcca

SEQ ID NO: 496 - RNA, 22 nt, Artificial
  anti-microRNA molecule
ugaagccgguugguagcuuuaa

SEQ ID NO: 497 - RNA, 22 nt, Artificial
  anti-microRNA molecule
ucaaggcuucaucaacaacgaa

SEQ ID NO: 498 - RNA, 22 nt, Artificial
  anti-microRNA molecule
uggacagcuauggccugaugaa

SEQ ID NO: 499 - RNA, 22 nt, Artificial
  anti-microRNA molecule
agcacaaacaaccaggccucca

SEQ ID NO: 500 - RNA, 22 nt, Artificial
  anti-microRNA molecule
agcuuugguaaccuagcuuuau

SEQ ID NO: 501 - RNA, 22 nt, Artificial
  anti-microRNA molecule
guucagaaucaugucgaaagcu

SEQ ID NO: 502 - RNA, 22 nt, Artificial
  anti-microRNA molecule
cggcuuucaacuaaugaucuca

SEQ ID NO: 503 - RNA, 22 nt, Artificial
  anti-microRNA molecule
acuagcuuucacgaugaucuca

SEQ ID NO: 504 - RNA, 22 nt, Artificial
  anti-microRNA molecule
acuggcuuucacgaugaucuca

SEQ ID NO: 505 - RNA, 22 nt, Artificial
  anti-microRNA molecule
uuacugaauuuauauggugcua

SEQ ID NO: 506 - RNA, 22 nt, Artificial
  anti-microRNA molecule
uacaauauuacauacuaccuca

SEQ ID NO: 507 - RNA, 22 nt, Artificial
  anti-microRNA molecule
acgacuuuucaaauacuuugua

SEQ ID NO: 508 - RNA, 22 nt, Artificial
  anti-microRNA molecule
acuguggcaaagcauucacuua

SEQ ID NO: 509 - RNA, 22 nt, Artificial
  anti-microRNA molecule
gcacaccugaaacuuugcucac

SEQ ID NO: 510 - RNA, 22 nt, Artificial
  anti-microRNA molecule
ggggcauucaaacaacauauca

SEQ ID NO: 511 - RNA, 22 nt, Artificial
  anti-microRNA molecule
guggcauucaccgcgugccuua

SEQ ID NO: 512 - RNA, 22 nt, Artificial
  anti-microRNA molecule
cgugaauucaugcagugccauu

SEQ ID NO: 513 - RNA, 22 nt, Artificial
  anti-microRNA molecule
ggaaaagauaaccagugucauu

SEQ ID NO: 514 - RNA, 22 nt, Artificial
  anti-microRNA molecule
cuccuggucgcacaacuaauac

SEQ ID NO: 515 - RNA, 22 nt, Artificial
  anti-microRNA molecule
cugccuguugaucacgagcuua

SEQ ID NO: 516 - RNA, 22 nt, Artificial
  anti-microRNA molecule
caccgcaguuaagaugcauuua

SEQ ID NO: 517 - RNA, 22 nt, Artificial
  anti-microRNA molecule
cccgcacaugcgcauugcucaa

SEQ ID NO: 518 - RNA, 22 nt, Artificial
  anti-microRNA molecule
aaaggguauucucgagcaauaa

SEQ ID NO: 519 - RNA, 22 nt, Artificial
  anti-microRNA molecule
ucaggccggggagagugcaaua

SEQ ID NO: 520 - RNA, 22 nt, Artificial
  anti-microRNA molecule
gcgucauuaccugacaguauua

SEQ ID NO: 521 - RNA, 22 nt, Artificial
  anti-microRNA molecule
gcuguucgagaauucucaggga

SEQ ID NO: 522 - RNA, 22 nt, Artificial
  anti-microRNA molecule
cugaauggcaucggaguacaaa

SEQ ID NO: 523 - RNA, 22 nt, Artificial
  anti-microRNA molecule
caguaccuauguguaguacaaa

SEQ ID NO: 524 - RNA, 22 nt, Artificial
  anti-microRNA molecule
caguacuuuuguguaguacaaa

SEQ ID NO: 525 - RNA, 22 nt, Artificial
  anti-microRNA molecule
agcgaagauuugggggccagua

SEQ ID NO: 526 - RNA, 22 nt, Artificial
  anti-microRNA molecule
gucauuucucgcaccuaccuca

SEQ ID NO: 527 - RNA, 22 nt, Artificial
  anti-microRNA molecule
cucgaagcaaaggccuacgcaa

SEQ ID NO: 528 - RNA, 22 nt, Artificial
  anti-microRNA molecule
auaucccgccgcgaucguaccg

SEQ ID NO: 529 - RNA, 22 nt, Artificial
  anti-microRNA molecule
uaccacuuuguacaaccaaaga

SEQ ID NO: 530 - RNA, 22 nt, Artificial
  anti-microRNA molecule
gagcuacuuggaggggaccaau

SEQ ID NO: 531 - RNA, 22 nt, Artificial
  anti-microRNA molecule
agcuccuacccgaaacauguaa

SEQ ID NO: 532 - RNA, 22 nt, Artificial
  anti-microRNA molecule
agaagagaauaggcucuaguca

SEQ ID NO: 533 - RNA, 22 nt, Artificial
  anti-microRNA molecule
gagcguuauccgugcacgugua

SEQ ID NO: 534 - RNA, 22 nt, Artificial
  anti-microRNA molecule
gcaacgcucaaaaguccuguga

SEQ ID NO: 535 - RNA, 22 nt, Artificial
  anti-microRNA molecule
ccaugccaacaguugacuguga

SEQ ID NO: 536 - RNA, 22 nt, Artificial
  anti-microRNA molecule
uaagagcggcaccacuacuuaa

SEQ ID NO: 537 - RNA, 22 nt, Artificial
  anti-microRNA molecule
uuaccugcggcacuacuacuua

SEQ ID NO: 538 - RNA, 22 nt, Artificial
  anti-microRNA molecule
uggucaguguuagugaggugug

SEQ ID NO: 539 - RNA, 22 nt, Artificial
  anti-microRNA molecule
cuacagucgcgaaagauuugca

SEQ ID NO: 540 - RNA, 22 nt, Artificial
  anti-microRNA molecule
guacagucuucuaugcauucca

SEQ ID NO: 541 - RNA, 22 nt, Artificial
  anti-microRNA molecule
ucacuggguacuccugauacuc

SEQ ID NO: 542 - RNA, 22 nt, Artificial
  anti-microRNA molecule
uaaaaggauuccucucaaaacc

SEQ ID NO: 543 - RNA, 22 nt, Artificial
  anti-microRNA molecule
ccagauuaggaugagauuuacu

SEQ ID NO: 544 - RNA, 22 nt, Artificial
  anti-microRNA molecule
uccuacaagaguucgacaucac

SEQ ID NO: 545 - RNA, 22 nt, Artificial
  anti-microRNA molecule
caccgugaaaacuaaaaagcua

SEQ ID NO: 546 - RNA, 22 nt, Artificial
  anti-microRNA molecule
guaucagaaaacaucgagaaac

SEQ ID NO: 547 - RNA, 22 nt, Artificial
  anti-microRNA molecule
cccauaacaacaaccacccgcc

SEQ ID NO: 548 - RNA, 22 nt, Artificial
  anti-microRNA molecule
aaauaccacccuuccucccuca

SEQ ID NO: 549 - RNA, 22 nt, Artificial
  anti-microRNA molecule
aagcuuugccaaagucuugccu

SEQ ID NO: 550 - RNA, 22 nt, Artificial
  anti-microRNA molecule
auugcagcagacacuucacggg

SEQ ID NO: 551 - RNA, 22 nt, Artificial
  anti-microRNA molecule
caaacugcuucuaauucuugcc

SEQ ID NO: 552 - RNA, 22 nt, Artificial
  anti-microRNA molecule
caaguuuugccagagucuugcc

SEQ ID NO: 553 - RNA, 22 nt, Artificial
  anti-microRNA molecule
aucuccacugcuacaucaugcc

SEQ ID NO: 554 - RNA, 22 nt, Artificial
  anti-microRNA molecule
cgaaugcuuucccacccggcga

SEQ ID NO: 555 - RNA, 22 nt, Artificial
  anti-microRNA molecule
cuuccaaacacccaugccuaca

SEQ ID NO: 556 - RNA, 22 nt, Artificial
  anti-microRNA molecule
agcagccgacacaguacgggca

SEQ ID NO: 557 - RNA, 22 nt, Artificial
  anti-microRNA molecule
gaauucuuccagugccauuaac

SEQ ID NO: 558 - RNA, 22 nt, Artificial
  anti-microRNA molecule
gcccuuaucaguucuccgucca

SEQ ID NO: 559 - RNA, 22 nt, Artificial
  anti-microRNA molecule
cccguuagugucggucacaaaa

SEQ ID NO: 560 - RNA, 22 nt, Artificial
  anti-microRNA molecule
gcgcgcuacuucagguaccuga

SEQ ID NO: 561 - RNA, 22 nt, Artificial
  anti-microRNA molecule
auaggccgggacaagugcaaug

SEQ ID NO: 562 - RNA, 22 nt, Artificial
  anti-microRNA molecule
aagaauugcguuuggacaauca

SEQ ID NO: 563 - RNA, 22 nt, Artificial
  anti-microRNA molecule
agagcacgguaugaaguuccua

SEQ ID NO: 564 - RNA, 22 nt, Artificial
  anti-microRNA molecule
gucguaccagauagugcauuua

SEQ ID NO: 565 - RNA, 22 nt, Artificial
  anti-microRNA molecule
aaacggacgaaagucccaccga

SEQ ID NO: 566 - RNA, 22 nt, Artificial
  anti-microRNA molecule
acagcugguugaaggggaccaa

SEQ ID NO: 567 - RNA, 22 nt, Artificial
  anti-microRNA molecule
uuaaugaguguggaucuaguca

SEQ ID NO: 568 - RNA, 22 nt, Artificial
  anti-microRNA molecule
acaaugcgacuacaaugcaccu

SEQ ID NO: 569 - RNA, 22 nt, Artificial
  anti-microRNA molecule
uuucauaugcaacguaaauaca

SEQ ID NO: 570 - RNA, 22 nt, Artificial
  anti-microRNA molecule
caaagagagcaauuccaugaca

SEQ ID NO: 571 - RNA, 22 nt, Artificial
  anti-microRNA molecule
aaagccuaguagaggcuagauu

SEQ ID NO: 572 - RNA, 22 nt, Artificial
  anti-microRNA molecule
cagaauuaccagcugauauuua

SEQ ID NO: 573 - RNA, 22 nt, Artificial
  anti-microRNA molecule
uggaaucaaguugcugacuuca

SEQ ID NO: 574 - RNA, 22 nt, Artificial
  anti-microRNA molecule
caaccagcuaaccacacugcca

SEQ ID NO: 575 - RNA, 22 nt, Artificial
  anti-microRNA molecule
uuggcauucaccgcgugccuua

SEQ ID NO: 576 - RNA, 22 nt, Artificial
  anti-microRNA molecule
augcuuugguaaucuagcuuua

SEQ ID NO: 577 - RNA, 22 nt, Artificial
  anti-microRNA molecule
agagcacgguauuaaguuccua

SEQ ID NO: 578 - RNA, 22 nt, Artificial
  anti-microRNA molecule
auagccgcugucacacgcacaa

SEQ ID NO: 579 - RNA, 22 nt, Artificial
  anti-microRNA molecule
gcacugauuucgaauggugcua

SEQ ID NO: 580 - RNA, 22 nt, Artificial
  anti-microRNA molecule
cacaaguucggauuuacggguu

SEQ ID NO: 581 - RNA, 22 nt, Artificial
  anti-microRNA molecule
gcaggccgggacuagugcaauu

SEQ ID NO: 582 - RNA, 22 nt, Artificial
  anti-microRNA molecule
gcacgaguguucggucuaguca

SEQ ID NO: 583 - RNA, 22 nt, Artificial
  anti-microRNA molecule
cgugcaaacgauuuucaacaca

SEQ ID NO: 584 - RNA, 22 nt, Artificial
  anti-microRNA molecule
acacaccugaaauuuugcucaa

SEQ ID NO: 585 - RNA, 22 nt, Artificial
  anti-microRNA molecule
ugugaauucucccagugccaag

SEQ ID NO: 586 - RNA, 22 nt, Artificial
  anti-microRNA molecule
augaaaugaaaucgacaugaaa

SEQ ID NO: 587 - RNA, 22 nt, Artificial
  anti-microRNA molecule
gcaggcuccacuuaaauauuua

SEQ ID NO: 588 - RNA, 22 nt, Artificial
  anti-microRNA molecule
aucagcuuucaaaaugaucuca

SEQ ID NO: 589 - RNA, 22 nt, Artificial
  anti-microRNA molecule
ccaguuuccugugaaaccuaaa

SEQ ID NO: 590 - RNA, 22 nt, Artificial
  anti-microRNA molecule
cagcuauuccgacaucuugcca

SEQ ID NO: 591 - RNA, 22 nt, Artificial
  anti-microRNA molecule
ucacauuuacaaauugagauua

SEQ ID NO: 592 - RNA, 22 nt, Artificial
  anti-microRNA molecule
agagcaccugaugaaguacaau

SEQ ID NO: 593 - RNA, 22 nt, Artificial
  anti-microRNA molecule
ucuacagcuagaauaccaaaga

SEQ ID NO: 594 - RNA, 22 nt, Artificial
  anti-microRNA molecule
uugagagucacuaaguaccuga

SEQ ID NO: 595 - RNA, 22 nt, Artificial
  anti-microRNA molecule
auacagcuaaaaucaccaaaga

SEQ ID NO: 596 - RNA, 22 nt, Artificial
  anti-microRNA molecule
ucacaaguuagggucucaggga

SEQ ID NO: 597 - RNA, 22 nt, Artificial
  anti-microRNA molecule
cgcucacucaaggagguuguga

SEQ ID NO: 598 - RNA, 22 nt, Artificial
  anti-microRNA molecule
cucacaguauaauccugugauu

SEQ ID NO: 599 - RNA, 22 nt, Artificial
  anti-microRNA molecule
cagcuaugccgacaucuugcca

SEQ ID NO: 600 - RNA, 22 nt, Artificial
  anti-microRNA molecule
uaggacaaacuuuacccagugc

SEQ ID NO: 601 - RNA, 22 nt, Artificial
  anti-microRNA molecule
aaaggccgggaagugugcaaua

SEQ ID NO: 602 - RNA, 22 nt, Artificial
  anti-microRNA molecule
ucaggccggugaaugugcaaua

SEQ ID NO: 603 - RNA, 22 nt, Artificial
  anti-microRNA molecule
ucaggccgucucaagugcaaua

SEQ ID NO: 604 - RNA, 22 nt, Artificial
  anti-microRNA molecule
ucgggcugugaaaagugcaaua

SEQ ID NO: 605 - RNA, 21 nt, Artificial
  anti-microRNA molecule
ccgaacuuauuggcucgaaua

SEQ ID NO: 606 - RNA, 22 nt, Artificial
  anti-microRNA molecule
gcuuucugagcaacaaucaaaa

SEQ ID NO: 607 - RNA, 22 nt, Artificial
  anti-microRNA molecule
cgccaguaagcggaaaaagaca

SEQ ID NO: 608 - RNA, 22 nt, Artificial
  anti-microRNA molecule
uggauaccaccagcuguguuca

SEQ ID NO: 609 - RNA, 22 nt, Artificial
  anti-microRNA molecule
ugagauaaacaaagcccaguga

SEQ ID NO: 610 - RNA, 22 nt, Artificial
  anti-microRNA molecule
cccaucaaagcuggcugugaua

SEQ ID NO: 611 - RNA, 22 nt, Artificial
  anti-microRNA molecule
ucaggauacauucaguauacgu

SEQ ID NO: 612 - RNA, 22 nt, Artificial
  anti-microRNA molecule
uacguauacugaagguauaccg

SEQ ID NO: 613 - RNA, 23 nt, Artificial
  2′-O-methyl microRNA molecule
gucaacaucagucugauaagcua

SEQ ID NO: 614 - RNA, 21 nt, Artificial
  2′-O-methyl antisense molecule
aaggcaagcugacccugaagu

SEQ ID NO: 615 - RNA, 21 nt, Artificial
  2′ O- methyl reverse seqeunce
ugaagucccagucgaacggaa

SEQ ID NO: 616 - DNA, 26 bp, Artificial
  2′-deoxy microRNA molecule
gtcaacatcagtctgataagctagcg

SEQ ID NO: 617 - DNA, 24 bp, Artificial
  2′-deoxy antisense molecule
aaggcaagctgaccctgaagtgcg

SEQ ID NO: 618 - DNA, 88 bp, Artificial
  primer
gaacaattgcttttacagatgcacatatcgaggtgaacatcacgtacgtcaacatcagtctgataagctatcggttggcagaagctat

SEQ ID NO: 619 - DNA, 90 bp, Artificial
  primer
ggcataaagaattgaagagagttttcactgcatacgacgattctgtgatttgtattcagcccatatcgtttcatagcttctgccaaccga

SEQ ID NO: 620 - DNA, 35 bp, Artificial
  primer
taatacgactcactatagaacaattgcttttacag

SEQ ID NO: 621 - DNA, 35 bp, Artificial
  primer
atttaggtgacactataggcataaagaattgaaga

SEQ ID NO: 622 - DNA, 33 bp, Artificial
  synthetic oligonucleotide molecule
ggcctcaacatcagtctgataagctaggtacct

SEQ ID NO: 623 - DNA, 33 bp, Artificial
  synthetic oligonucleotide molecule
ggccaggtacctagcttatcagactgatgttga